(19) United States
(12) Patent Application Publication
(10) Pub. No.: US 2021/0310788 A1
(43) Pub. Date: Oct. 7, 2021

(54) APPARATUS AND METHODS FOR SPECKLE REDUCTION AND STRUCTURE EXTRACTION IN OPTICAL COHERENCE TOMOGRAPHY

(71) Applicant: THE REGENTS OF THE UNIVERSITY OF CALIFORNIA, Oakland, CA (US)

(72) Inventors: Robert J. Zawadzki, Sacramento, CA (US); Edward N. Pugh, JR., Davis, CA (US); Pengfei Zhang, Davis, CA (US)

(73) Assignee: THE REGENTS OF THE UNIVERSITY OF CALIFORNIA, Oakland, CA (US)

(21) Appl. No.: 17/168,043

(22) Filed: Feb. 4, 2021

Related U.S. Application Data
(63) Continuation of application No. PCT/US2019/046055, filed on Aug. 9, 2019.
(60) Provisional application No. 62/716,689, filed on Aug. 9, 2018.

(51) Int. Cl. G01B 9/02; G06T 7/246

(52) U.S. Cl. CPC G01B 9/02082; G06T 2207/10101; G01B 9/02091; G06T 7/248

(57) ABSTRACT

Systems, apparatus and methods that modulate the phase inside the imaging system pupil aperture with a segmented deformable mirror, spatial light modulator (SLM), or liquid deformable lens (LDL) to produce minor perturbations in the point spread function (PSF) and create un-correlated speckle patterns between B-scans.

### CROSS-REFERENCE TO RELATED APPLICATIONS

This application claims priority to, and is a 35 U.S.C. § 111(a) continuation of, PCT international application number PCT/US2019/046055 filed on Aug. 9, 2019, incorporated herein by reference in its entirety, which claims priority to, and the benefit of, U.S. provisional patent application Ser. No. 62/716,689 filed on Aug. 9, 2018, incorporated herein by reference in its entirety. Priority is claimed to each of the foregoing applications.

The above-referenced PCT international application was published as PCT International Publication No. WO 2020/033920 A1 on Feb. 13, 2020, which publication is incorporated herein by reference in its entirety.

### STATEMENT REGARDING FEDERALLY SPONSORED RESEARCH OR DEVELOPMENT

This invention was made with Government support under Grant Nos. EY002660 and EY026556, awarded by the National Institutes of Health, and under Grant No. IIP-1650588, awarded by the National Science Foundation. The Government has certain rights in the invention.

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to Optical Coherence Tomography (OCT), and more particularly to speckle reduction for OCT.

### 2. Background Discussion

Speckle is a long-standing issue in imaging technologies that use coherent light sources. Speckle arises from the interference between light scattered by random distributed scatterers inside the system point-spread function (PSF), and observed as voxel-to-voxel intensity fluctuations in the image. Although speckle is potentially useful information about the dynamics of sample microstructure, in most applications it acts as a major noise source that degrades image quality.

Optical coherence tomography (OCT) is a volumetric imaging technology and has been adapted for use in many biomedical applications. However, as a method dependent on the coherent properties of light, OCT images suffer from speckle noise which imposes significant limitations on the diagnostic capabilities of the system.

Many approaches have been taken to suppress speckle, including generation of multiple images by various means with uncorrelated speckle patterns, followed by averaging. A weakness of these methods is that the number of uncorrelated speckle patterns that can be created is typically small, thereby limiting speckle suppression by averaging. Speckle reduction methods using digital post-processing have also been proposed. However, digital post-processing usually reduces speckle by spatial averaging or filtering, which necessarily reduces image resolution. Recently, it was shown that simple averaging of suitably numerous, well aligned images can reduce speckle for in vivo imaging, and it was hypothesized that subcellular motility of scatterers was responsible for varying the speckle pattern between frames. Because this latter method relies on time-dependent variation in the sample microstructure, it is inherently passive and dependent on the underlying time course of the mobile scatterers.

As a way of potentially overcoming the limitations of passive averaging, speckle modulating OCT (APM-OCT) was recently developed. By introduction of a ground-glass diffuser in the external optical path, the method generates random, time-varying changes in the sample beam. It is hypothesized that the approach introduces axial phase variation in the imaging plane. However, this phase variation is not directly under experimenter control, and the phase shift cannot be readily repeated. In contrast, as characterized in classical optical theory and applied in adaptive optics (AO) imaging, the phase can be precisely controlled by manipulation of the wavefront corrector at the system pupil aperture, and this suggests the possibility of using AO technology to create a method for speckle suppression that would be readily controllable and broadly applicable to OCT.

### BRIEF SUMMARY

An aspect of the present disclosure is apparatus and methods that use aperture phase modulation (APM) with adaptive optics (AO) for speckle reduction and structure extraction in optical coherence tomography (OCT).

Speckle is an inevitable consequence of the use of coherent light in OCT and often acts as noise that obscures micro-structures of biological tissue. To address that problem, a system and method of the present disclosure provides speckle noise suppression in a manner that is intrinsically compatible with AO in an OCT system. In one embodiment, the method of the present disclosure provides the step of modulating the phase inside the imaging system pupil aperture with a segmented deformable mirror, spatial light modulator (SLM), or liquid deformable lens (LDL) to produce minor perturbations in the point spread function (PSF) and create un-correlated speckle patterns between B-scans. Averaging techniques may then be used to wash out the speckle but maintain the structures.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 shows a schematic diagram of an exemplary system for performing aperture phase modulation (APM) with adaptive optics (AO) for speckle reduction and structure extraction in optical coherence tomography (OCT) via a segmented deformable mirror as phase modulator in accordance with the present description.

FIG. 2A is a top schematic view of a flat mirror configuration for the segmented deformable mirror of FIG. 1.

FIG. 2B is a top schematic view of a mirror configuration with mirror segments randomly actuated pistons for the segmented deformable mirror of FIG. 1.

FIG. 3 shows a histogram of the random mirror displacements for 100 mirror segments of the deformable mirror of FIG. 1, wherein the displacement range was 1 μm (0±0.5 μm).

FIG. 4 shows a schematic diagram of an exemplary system for performing aperture phase modulation (APM) with adaptive optics (AO) for speckle reduction and structure extraction in optical coherence tomography (OCT) via a reflective spatial light modulator (SLM) as phase modulator in accordance with the present description.

FIG. 5 shows a schematic diagram of an exemplary system for performing aperture phase modulation (APM) with adaptive optics (AO) for speckle reduction and structure extraction in optical coherence tomography (OCT) via a transmissive SLM or liquid deformable lens (LDL) as phase modulator in accordance with the present description.

FIG. 6A is a plot illustrating a searching process over a single Zernike mode as an example.

FIG. 6B is a plot of enface image brightness changes after each Zernike mode search for the dashed box illustrated in the image of FIG. 8

FIG. 7 shows optimal Zernike coefficients and the mirror shape (inset).

FIG. 8 shows B-scan images before and after AO-correction.

FIG. 9A and FIG. 9 B show enface images before and after AO-correction, respectively.

FIG. 10A shows a schematic diagram for AO-OCT, wherein the DM elements are flattened for resolution target imaging or optimized for aberration-corrected retinal imaging.

FIG. 10B shows a schematic diagram for APM-AO-OCT wherein the DM elements are articulated to a random displacement pattern over the top surface of the mirror.

FIG. 10C illustrates a schematic diagram for an alternate B-scan saving mode, wherein a certain number (N) of OCT and (N) APM-OCT B-scans are acquired in turns to ensure strict comparison, while the y-scanner is not moving.

FIG. 10D illustrates a schematic diagram for an alternate volume saving mode, wherein a certain number (N) of OCT and APM-OCT B-scans are acquired in turns (sequential, repeating order) to ensure strict comparison, while the y-scanner is moving one step right before each acquisition block (N-OCT+N-APM-OCT).

FIG. 11A shows images of individual (1 . . . N) and 100-frames-averaged OCT B-scans.

FIG. 11B shows images of individual (1 . . . N) and 100-frames-averaged APM-OCT B-scans.

FIG. 12A shows a schematic representation of the in-focus 3D OCT PSF (ellipse).

FIG. 12B illustrates a configuration when the DM 12A is in a flat mode.

FIG. 12C illustrates a configuration when the DM 12A is configured as ‘random’ mode, a dynamic PSF selects different scatterer sets.

FIG. 13A is a printed 1951 USAF resolution test target image.

FIG. 13B shows an image of the B-scan of the target in FIG. 13A averaged from an ensemble of 100 scans taken with no DM modulation, and exhibits speckle noise.

FIG. 13C shows an image of the B-scan of the target in FIG. 13A averaged from an ensemble of 100 scans taken with each DM facet 24 displaced randomly over a 0.3 μm range (0±0.15 μm), and shows strongly reduced speckle.

FIG. 14A shows speckle contrast as a function of the averaged B-scans numbers for different random mirror displacement ranges, with the gray-scale bar specifying the displacement range.

FIG. 14B shows a plot for curves comprising speckle contrast (from the data indicated by the arrow in FIG. 14A) and resolution (dashed curve) compared in averaged B-scans as a function of the mirror displacement range.

FIG. 15A shows a plot of average intensity of 1000 APM-OCT B-scans plotted in descending order (the mirror displacement range was 0.3 μm), wherein the left inset image shows a covariance analysis of the top 100 mirror configurations, and the right inset image shows an enface test target image with arrows indicating the B-scan locations for the plots in FIG. 15B with the same order (from top to bottom).

FIG. 15B shows APM-OCT signals from 1000 B-scans with the same mirror configurations in FIG. 15A.

FIG. 15C shows a plot illustrating speckle contrast comparison for 100-frames-averaged APM-OCT images obtained with random and the selected “top 100” mirror configurations from the shaded region marked in FIG. 15A.

FIG. 15D illustrates resolution plotted as a function of DM displacement range for different configurations: random (upper line), the top 10% (circles), or the top 2% (lower line).

FIG. 16A-FIG. 16M illustrate a comparison of the efficiency of the averaging of APM-AO-OCT vs AO-OCT results in reducing speckle and revealing novel cellular structure in vivo.

FIG. 17A-FIG. 17J illustrate visualization of cellular scale structures in retinal layers with in vivo volumetric APM-AO-OCT.

FIG. 18A shows a projection of 1000 APM-AO-OCT PSFs produced by mirror segment displacement range of 0.3 μm.

FIG. 18B shows a projection of “top 100” PSFs from the sample of 1000 presented in FIG. 18A.

FIG. 18C shows a projection of 1000 AO-OCT PSFs (no DM modulation); the 1000 PSFs were indistinguishable from one another.

FIG. 18D shows an average of the 1000 APM-AO-OCT PSFs presented in FIG. 18A.

FIG. 18E shows an average of the “top 100” APM-AO-OCT PSFs presented in FIG. 18B.

FIG. 18F shows line profiles of the averaged PSFs.

FIG. 19 shows a flowchart of an embodiment of data acquisition according to the presented technology.

FIG. 20 shows a flowchart of an embodiment of OCT data processing and post-processing according to the presented technology.

FIG. 21 shows a flowchart of an embodiment of random number generation according to the presented technology.

FIG. 22 shows a flowchart of an embodiment of searching for optimum sets of PSFs for APM-AO-OCT for a given sample according to the presented technology.

FIG. 23 shows a flowchart of an embodiment of a method to acquire interlaced B-scans with AO-OCT and APM-AO-OCT B-scans according to the presented technology.

FIG. 24 shows a flowchart of an embodiment of a method to extend APM-AO-OCT interlaced B-scan acquisition to volumetric data acquisition by acquiring Serial B-scans and build OCT volume from that (slow data acquisition or static sample) according to the presented technology.

FIG. 25 shows a flowchart of an embodiment of a method to extend APM-AO-OCT interlaced B-scan acquisition to volumetric data acquisition by acquiring Serial Volumes and build APM-AO-OCT interlaced volume from that (fast data acquisition or moving sample) according to the presented technology.

FIG. 26 shows a flowchart of an embodiment of a method to deform segmented wavefront correctors that allows maintained lateral resolution while varying PSF according to the presented technology for further optimization.

FIG. 27 shows a flowchart showing an embodiment of two registration methods to reduce speckle by averaging optimized set of APM-AO-OCT B-scans according to the presented technology.

### DETAILED DESCRIPTION

The core of AO-enhanced imaging is the active control of the wavefront at the system aperture, a controlled implementation mostly by means of a phase modulating element in the form of a deformable mirror (DM), spatial light modulators (SLMs) or liquid deformable lens (LDL) to optimize the wavefront over the pupil to allow the system to operate at diffraction-limited performance. The systems and methods of the present description take advantage of this control to create a novel method for speckle noise reduction—aperture phase modulation AO-OCT (APM-AO-OCT).

In one embodiment, the system and method employ sub-micron piston modulations of the DM segments to introduce random phase variation for all segments in both spatial and temporal directions. The underlying mechanism is based on the premise that the modulations of DM mirror segments about their AO-optimized positions slightly alter the PSF, randomizing over samples the contributions from different scatterers to create uncorrelated speckle pattern, so that averaging can efficiently reduce the speckle. The inherent conflict between speckle noise reduction and preservation of signal resolution and strength is addressed by determining an optimum mirror segment displacement range. A relatively small subset of the total set of mirror configurations is identified within this range that maximally reduce speckle while preserve resolution and signal strength.

A. System and Methods

1. AO-OCT System Configuration

The adaptive optics (AO) systems of the present description utilize a phase modulating element (e.g. a deformable mirror (DM), spatial light modulator (SLM) or liquid deformable lens (LDL)) is placed in an optical plane conjugate with the pupil aperture to correct aberrations of the cornea and lens.

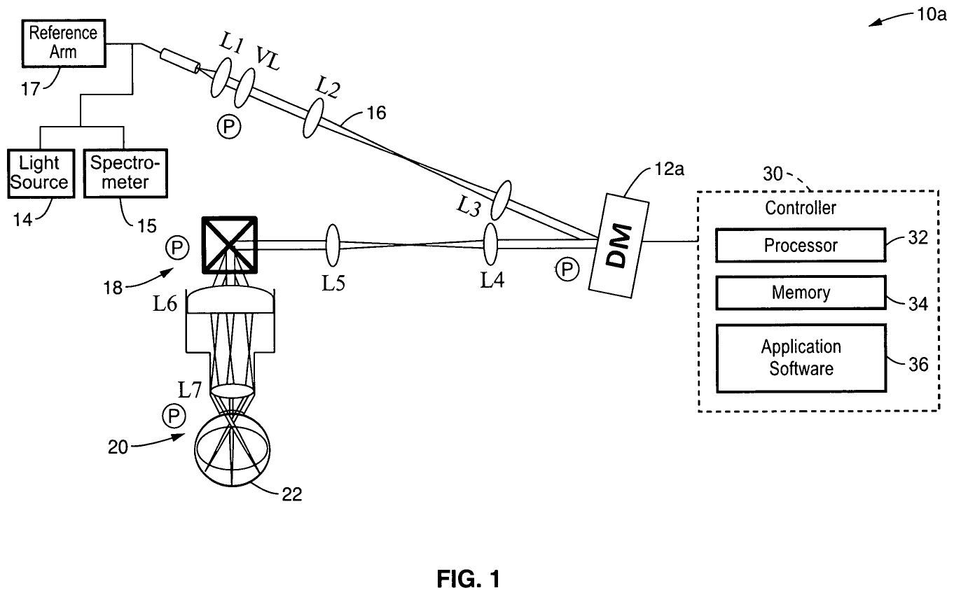

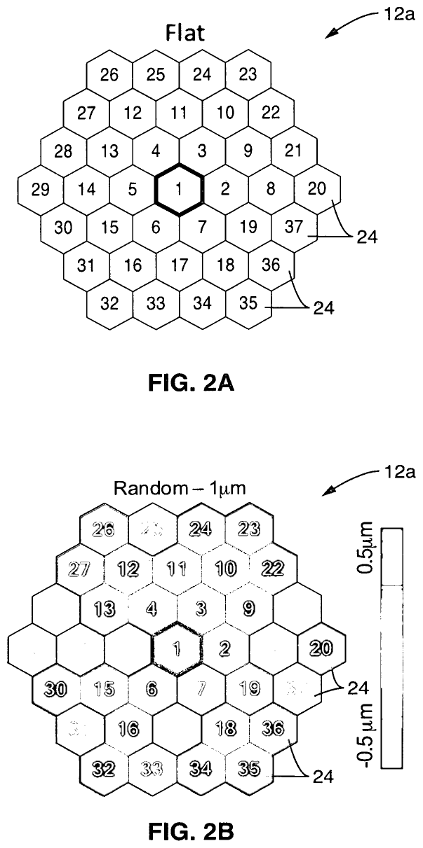

FIG. 1 shows a schematic diagram of an exemplary system 10a for performing aperture phase modulation (APM) with adaptive optics (AO) for speckle reduction and structure extraction in optical coherence tomography (OCT) via a segmented deformable mirror (DM 12a) as phase modulator. The DM 12a (e.g. PTT111, IRIS AO, Inc.) has 37 segments (see segments 24 in FIG. 2A and FIG. 2B) that are independently moveable via 111 actuators (not shown, 3 actuators per segment) to independently control the displacement/piston, tip and tilt of the segments 24. In one embodiment, each of the segments 24 are hexagonal and have a 0.7 mm pitch size. The DM 12a segments 24 have nanometer level displacement resolution (z-offset of the mirror surface) with a working range of [−2, 2] μm. As illustrated in FIG. 2A, When the DM 12a operates in ‘flat’ mode, the displacements of all segments are zero. FIG. 2B illustrates each segment 24 of the DM 12a independently controlled to operate in a ‘random’ displacement mode.

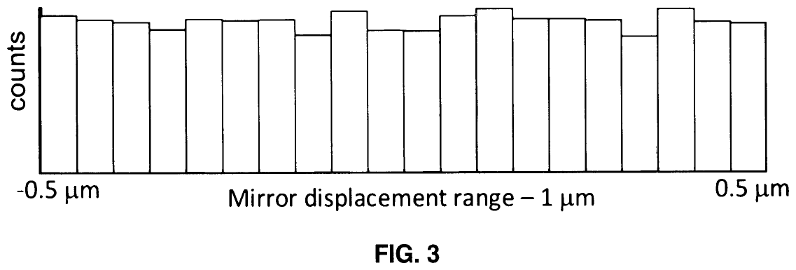

FIG. 3 shows a histogram of the random mirror displacements for 100 times running the deformable mirror of FIG. 1, wherein the displacement range was 1 μm (0±0.5 μm).

Referring back to FIG. 1, the segments 24 of DM 12a are coupled to a controller 30 comprising a processor 32, memory 34 and application software 36 stored in memory and executable on processor 32 for individually controlling DM 12a. In one embodiment, controller 30 comprises a computer, server or other processing device configured for executing application software 36, which may comprise instructions in the form of code for operating the DM 12a and/or image processing techniques detailed below.

With respect to the sample arm illustrated in FIG. 1, beam 16 emitted from light source 14 is modified by lenses L1, L2, L3, and variable focus length liquid lens VL prior to illuminating DM 12a (e.g. with a beam size of 3.5 mm to just fully cover the mirror 12a). After being reflected off DM 12a, beam passes through lenses, L4, L5, galvanometer scanner 18, and lenses L6 and L7 prior to being output 20 at eye 22 (e.g. mouse eye in experiments). P denotes optical planes conjugate with the pupil.

In one embodiment, the lenses used in the sample arm are VIS-NIR coated achromatic lenses (400-1000 nm, Edmunds Optics, key parameters are shown in Table S1), the light source 14 comprises a super-luminescent diode SLD (e.g. T-870-HP, Superlum, ranged from [780, 960] nm and centered at 870 nm) served as the light source for NIR OCT with a power at eye pupil of 900 μW; A customized spectrometer 15 with 2048 pixels was used to acquire the OCT spectra.

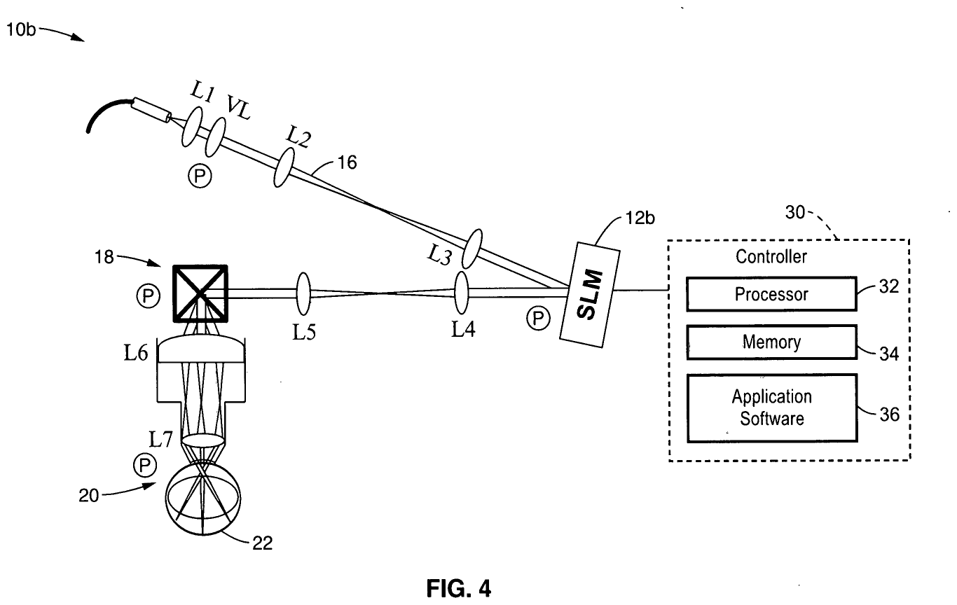

FIG. 4 shows a schematic diagram of an exemplary system 10b for performing aperture phase modulation (APM) with adaptive optics (AO) for speckle reduction and structure extraction in optical coherence tomography (OCT) via a reflective spatial light modifier (SLM)12b as phase modulator.

The SLM 12b phase modulator is coupled to a controller 30 comprising a processor 32, memory 34 and application software 36 stored in memory and executable on processor 32 for individually controlling SLM 12b.

With respect to the sample arm illustrated in FIG. 4, beam 16 emitted from light source 14 is modified by lenses L1, L2, L3, and variable focus length liquid lens VL prior to illuminating SLM 12b. After being reflected off SLM 12b, beam 16 passes through lenses, L4, L5, galvanometer scanner 18, and lenses L6 and L7 prior to being output 20 at eye 22 (e.g. mouse eye in experiments). P denotes optical planes conjugate with the pupil.

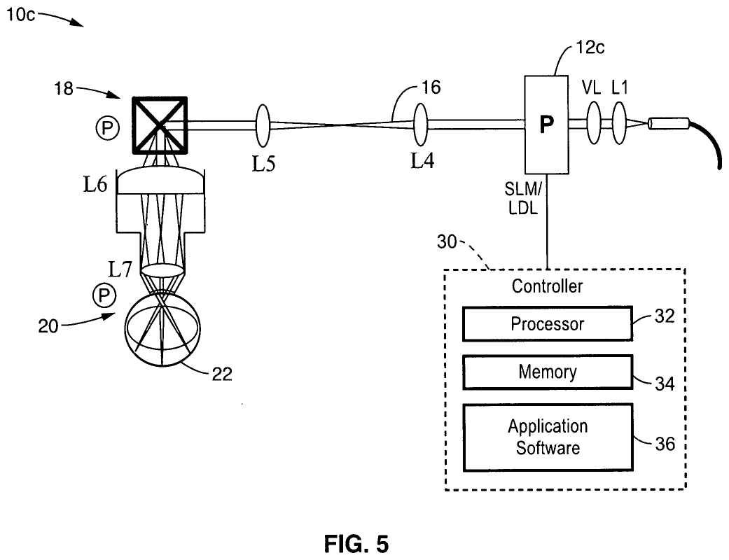

FIG. 5 shows a schematic diagram of an exemplary system 10c for performing aperture phase modulation (APM) with adaptive optics (AO) for speckle reduction and structure extraction in optical coherence tomography (OCT) via a transmissive SLM or liquid deformable lens (LDL) 12c as phase modulator.

The transmissive SLM/LDL12c phase modulator is coupled to a controller 30 comprising a processor 32, memory 34 and application software 36 stored in memory and executable on processor 32 for individually controlling SLM 12b.

With respect to the sample arm illustrated in FIG. 4, beam 16 emitted from light source 14 is modified by lenses L1, L2, L3, and variable focus length liquid lens VL prior to illuminating transmissive SLM/LDL12c. After being transmitted through transmissive SLM/LDL12c, beam 16 passes through lenses, L4, L5, galvanometer scanner 18, and lenses L6 and L7 prior to being output 20 at eye 22 (e.g. mouse eye in experiments). P denotes optical planes conjugate with the pupil.

2. Data Acquiring, Post-Processing and Quantification

OCT spectra were acquired at a 100 kHz A-scan rate using customized Labview software. Each B-scan comprised 550 A-scans, resulting in a B-scan rate of 30 Hz that included data acquisition, display and storage. Post-processing was implemented by customized Matlab™ code with standard functions including DC subtraction, dispersion compensation, wavelength-to-k-space interpolation, Hann windowing, and FFT. The results were then further processed by averaging or other analysis as indicated. The raw spectrum of each A-scan acquired with OCT and APM-OCT was processed in an exact same way to create images in the spatial domain for comparison.

A metric, normalized speckle contrast (NSC) was used to quantified compare the speckle noise suppression effect between images. It is defined as: the standard deviation (s.d.) of the image intensity in a given region divided by the mean image intensity of the same region. For concise purpose, speckle contrast, instead of its full name, was used in the main text.

The registration of in vivo imaging B-scans was done either by ImageJ TurboReg/StackReg plugin (for B-scan average), or the phase variance OCT software developed to do intensity averaging and/or blood vessel map extraction (for volume data average).

3. Wavefront Sensor-less (WFSL) Adaptive Optics Aberration Correction

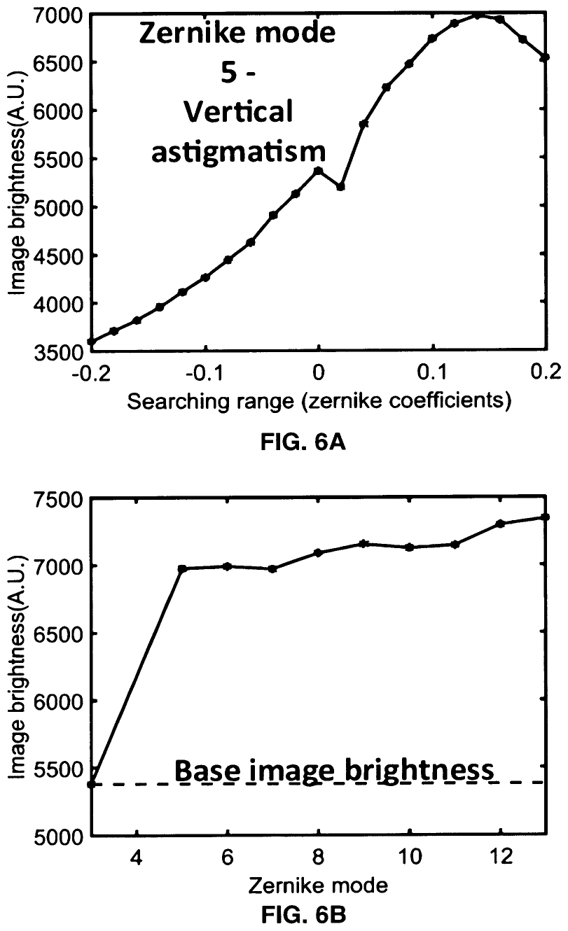

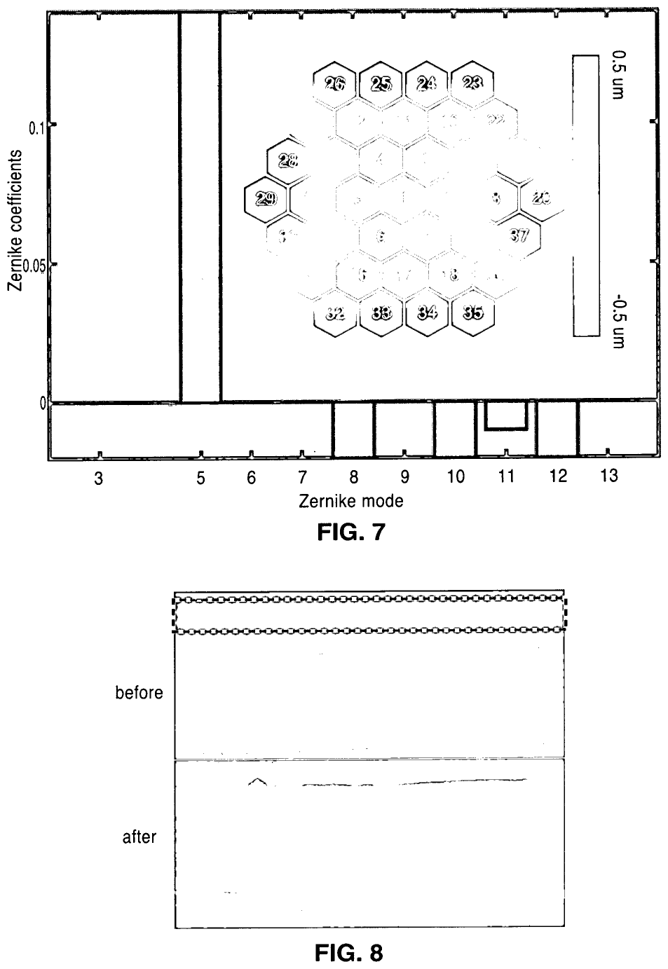

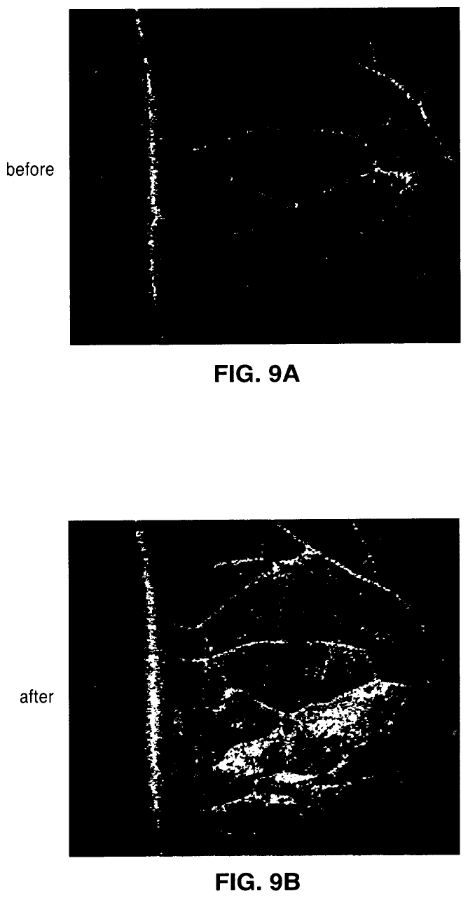

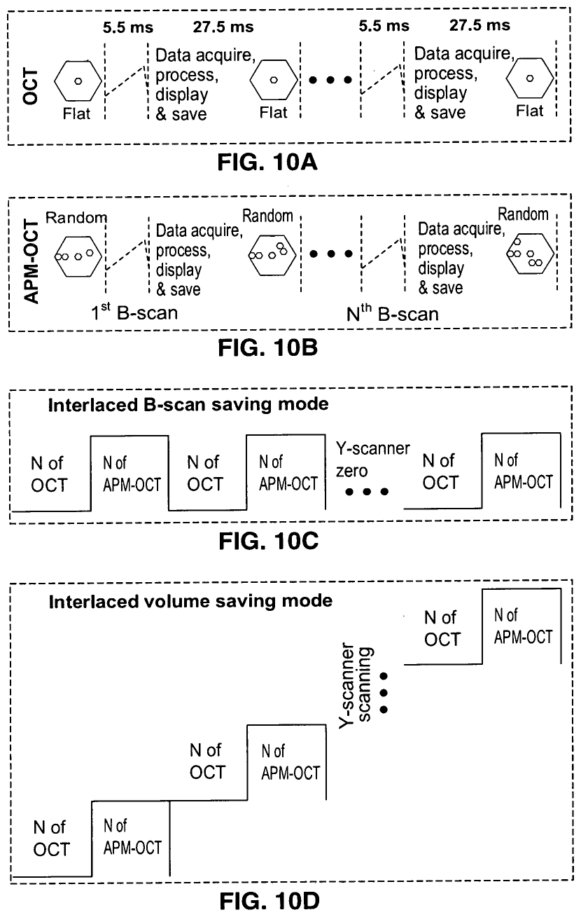

The image beam 20 at the eye pupil 22 has a diameter of 0.93 mm (Table 1), a size for which the ocular aberration is non-negligible. The eye's aberrations were first corrected using wavefront sensor-less (WFSL) aberration correction software with an image intensity-based searching, as shown in process illustrated in FIG. 6A through FIG. 9B. The software automatically calculates the brightness in a user-defined region of interest (ROI) layer, while varying the amplitudes of the DM in Zernike space (ANSI standard) over a search range. FIG. 6A is a plot illustrating a searching process over a single Zernike mode as an example. FIG. 6B is a plot of enface image brightness changes after each Zernike mode search for the dashed box illustrated in the image of FIG. 8. FIG. 7 shows optimal Zernike coefficients and the mirror shape (inset). FIG. 8 shows B-scan images before and after AO-correction. FIG. 9A and FIG. 9 B show enface images before and after AO-correction, respectively.

After the search process found the optimal mirror configuration for correcting the aberrations of the individual eye, the mirror configuration was loaded into the Labview-based data acquisition software.

4. Aperture Phase Modulation

In an optimized AO system, the DM defines a wavefront across the system aperture to correct aberrations, so as to approach diffraction-limited performance for the system NA, resulting in the most compact point-spread function possible for that NA. The aperture phase distribution was modulated about its optimum AO configuration by random displacements of the mirror segments using a uniform distribution centered on zero, with displacement ranges from 0 (no displacement) to 1.0 μm (0±0.5 μm). Histogram analysis of the mirror segments illustrate the uniform distribution of the displacements (FIG. 3). Covariance analysis of the mirror position matrix after 100 trials showed that the mirror segment displacements are uncorrelated.

5. 3D PSF of the AO-OCT System

In a scanning imaging system, the 3D distribution of power at the focal point in the sample defines the system's point-spread function. For diffraction-limited systems employing non-coherent light sources and having a circular aperture, the 3D PSF has an analytic form that can be approximated by a 3D ellipsoid. In OCT, which relies on partially coherent light for interferometry, beam propagation into the sample is governed by the NA of the system in the same manner as for non-coherent light, but the axial direction was further sectioned by the coherence length which is inversely proportional to source bandwidth. In the AO-OCT system used here the PSF has a calculated axial (coherence) length of ˜2.5 μm (in tissue, assuming a refraction index of 1.35). In OCT the sampling unit is the A-scan, which provides an axial profile of the backscattering light along the beam propagation axis. While the coherence length of the PSF is invariant with A-scan depth, the lateral (x-, y-) width of the PSF varies according to the NA, being wider away from the center focus. This lateral variation can be particularly notable in AO-OCT, where higher NA is employed, diminishing both the lateral resolution and the power density (imaging brightness) at axial distances away from the center focal plane.

6. Timing and Scanning Protocol

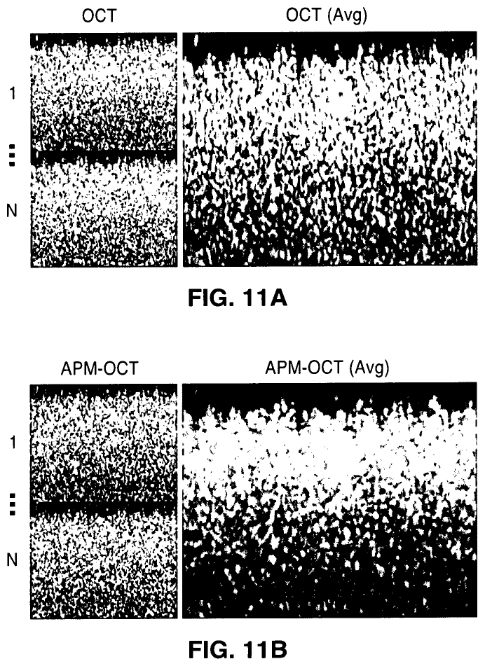

FIG. 10A through FIG. 10D illustrate a timing and scanning protocol for NIR-OCT in accordance with the present description. Referring to the schematic diagrams of FIG. 10A and FIG. 10B, the configuration of the moveable segments 24 of DM 12a was modified immediately prior to each B-scan. For AO-OCT illustrated in FIG. 10A, the elements 24 of DM 12a were flattened (FIG. 2A) for resolution target imaging or optimized for aberration-corrected retinal imaging. For APM-AO-OCT illustrated in FIG. 10B, the DM 12a elements 24 were articulated to generate a random displacement pattern (FIG. 2B) over the top surface specifically deviating from the optimal mirror shape for AO-OCT. If using an SLM 12b or 12c as in FIG. 4 and FIG. 5 (liquid crystals in SLM are birefringent), this random displacement pattern may be affected by applying a voltage to the cells to change the effective refractive index seen by the incident wave, and thus the phase retardation of the reflected wave in each SLM pixel. The approach with an LDL 12C (FIG. 5) is similar to a DM with many actuators to control the wavefront phase, and involves each actuator in the LDL 12C to perform certain actions to introduce proper phase modulation.

FIG. 10C illustrates a schematic diagram for an alternate B-scan saving mode, wherein a certain number (N) of OCT and (N) APM-OCT B-scans are acquired in turns to ensure strict comparison, while the y-scanner is not moving. In one exemplary mode, the B-scan based comparison between AO-OCT and APM-AO-OCT entails the x-scanner repeatedly scanning the same line on the sample, where the number of N (N=100 for ex vivo imaging, N=1 for in vivo imaging) AO-OCT and APM-AO-OCT scans were acquired in sequential, repeating order.

FIG. 10D illustrates a schematic diagram for an alternate volume saving mode, wherein a certain number (N) of OCT and APM-OCT B-scans are acquired in turns (sequential, repeating order) to ensure strict comparison, while the y-scanner is moving one step right before each acquisition block (N-OCT+N-APM-OCT). In one mode involving an enface comparison, a number of N (N=20, 50 or 100 for ex vivo imaging, N=50 for in vivo imaging) OCT and APM-OCT B-scans were acquired in the same location one after one, then the y-scanner moved to the next location to repeat the previous process until it covered the ROI.

In one embodiment, a N/30 (30 Hz B-scan rate) second difference between acquisition of AO-OCT and APM-AO-OCT data sets is performed to ensure strict comparison.

C. Results

1. Effect of Aperture Phase Modulation and Mechanism of Speckle Noise Reduction

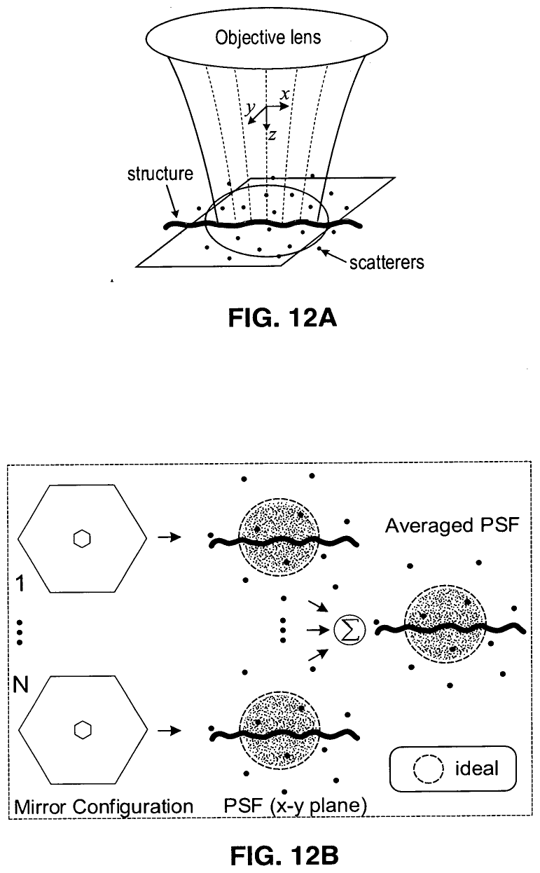

As previously detailed, speckle noise in OCT images arises from the interference between scattering light from different scatterers within the PSF and is observed as voxel-to-voxel intensity fluctuations in the image. In a single OCT B-scan of a Lambertian target (e.g. solid cylinder), the speckle pattern predominates to the extent that no structure can be discerned below the surface. Averaging 100 B-scans with unchanged DM configurations does little to suppress the speckle since the speckle pattern doesn't change, as dictated by physics, given that the sample and the underlying scatterers are immobile for non-biological sample (FIG. 11A).

The OCT imaging system has a deformable mirror (DM) whose actuators have a rapid response time, and so afford the possibility of manipulating the wavefront phase at the system aperture. If, prior to the collection of each B-scan, the DM mirror facets are randomly displaced a sub-micron distance, the speckle pattern changes between B-scans, further averaging will suppress the speckle (FIG. 11B).

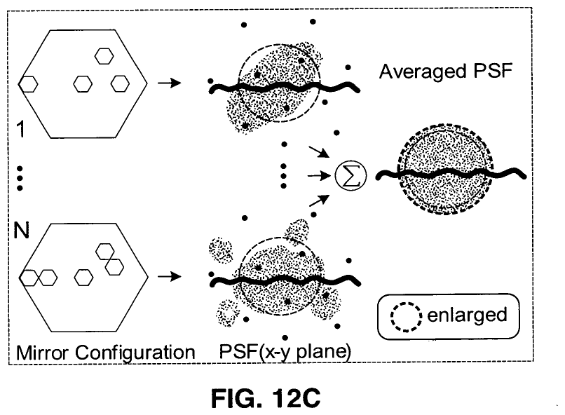

FIG. 12A shows a schematic representation of the in-focus 3D OCT PSF (ellipse).

FIG. 12B illustrates a configuration when the DM 12A is in a flat mode, a static PSF always selects same scatterer set. For non-living tissue, when the DM of the AO-imaging system is optimized, the PSF realizes its most compact form in the sample (PSF, x-y plane) and does not change, so that the scatterer set sample by the PSF is always same. This results in an unchanged speckle pattern, explaining why the average B-scan is very similar to any individual scan.

FIG. 12C illustrates a configuration when the DM 12A is configured as ‘random’ mode, a dynamic PSF selects different scatterer sets. Thus, random displacements of the DM segments 24 from their optimum positions alter the wavefront phase across the aperture, resulting in a PSF that is distorted from the optimum to varying in shapes, intensity distributions and/or extents (PSF, x-y plane). This altered PSF will probe a different set of scatterers which creates un-correlated speckle pattern between B-scans, while still including a portion of structures (FIG. 12C, thick wavy line, larger than the PSF in either of 3 dimensions) that was sampled by the undistorted PSF. Averaging over a population of B-scans taken with different DM patterns can thus reduce speckle while preserving signal from the structures.

With respect to APM-OCT there is an inherent conflict between the goal of reducing speckle noise and that of maintaining maximal image resolution. Also, the potential number of DM configurations is vast: for a mirror with 37 segments and a merely 11-step distribution over the displacement range, the total number of possible configurations is very large (11³⁷). Finally, with respect to reduced signal intensity, a preferred implementation of APM-OCT as a method of speckle noise reduction provides an efficient way of selecting a manageable subset of the mirror configurations that also resolves the conflict between speckle noise reduction, and preservation of resolution and signal strength.

2. Finding the DM Displacement Range That Both Reduces Speckle And Preserves Resolution

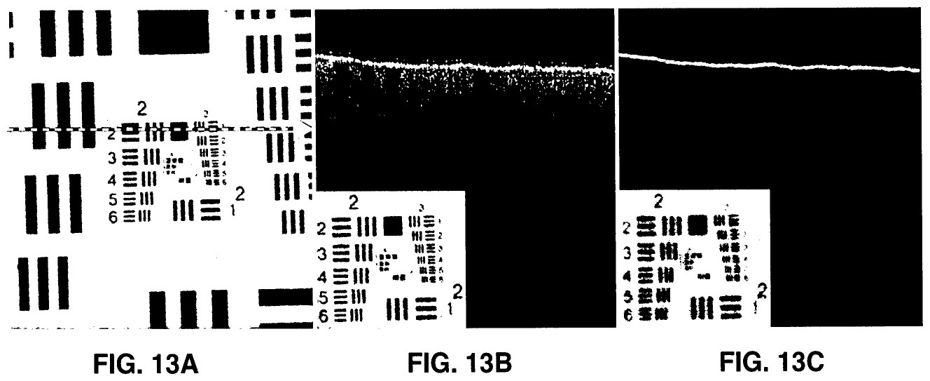

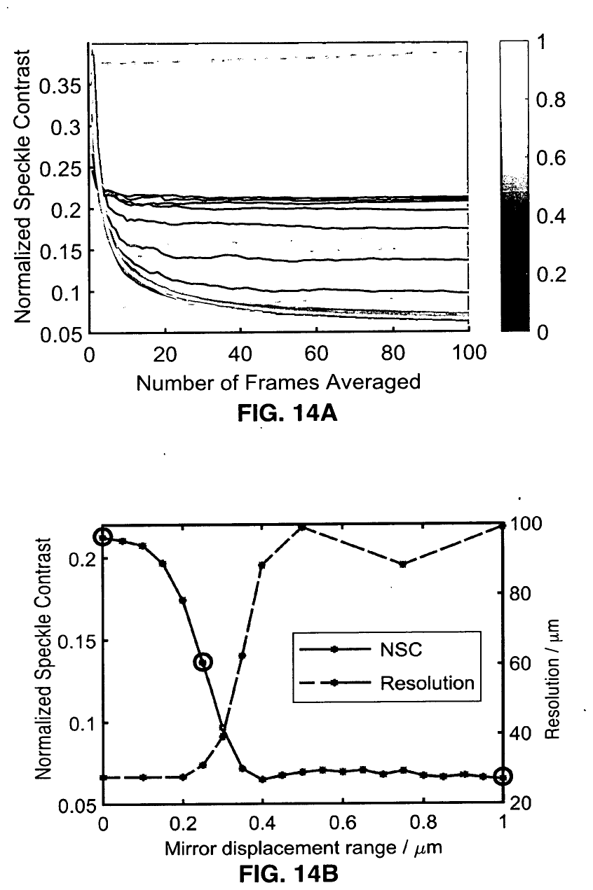

FIG. 13A through FIG. 14B illustrate a process for finding an optimal displacement range for minimizing speckle while preserving resolution. OCT imaging was performed on a printed 1951 USAF resolution test target shown in FIG. 13A (Newport, Irvine, Calif., U.S.). The dashed line in FIG. 13A indicates the OCT B-scans shown in FIG. 13B and FIG. 13C.

FIG. 13B shows an image (enface projection is inset) of the B-scan of the target in FIG. 13A averaged from an ensemble of 100 scans taken with no DM modulation (flat DM 12a). The image of FIG. 13B exhibits significant speckle noise.

FIG. 13C shows an image (enface projection is inset) of the B-scan of the target in FIG. 13A averaged from an ensemble of 100 scans taken with each DM facet 24 displaced randomly over a 0.3 μm range (0±0.15 μm). The image of FIG. 13C exhibits strongly reduced speckle.

FIG. 14A shows speckle contrast as a function of the averaged B-scans numbers for different random mirror displacement ranges, with the gray-scale bar specifying the displacement range. The dependence of speckle noise reduction on the number of averaged B-scan and mirror displacement range was quantified by calculating the normalized speckle contrast. Here the displacement ranges were varied from 0 (no displacement) to 1.0 μm with a uniform distribution centered on zero. Speckle contrast rapidly declined with the increased displacement range and/or number of B-scan averaged, approaching an asymptotic value.

Referring now to FIG. 14B, image resolution loss and speckle contrast reduction from these experiments were then compared as a function of the DM displacement range. FIG. 14B shows a plot for curves comprising speckle contrast (from the data indicated by the arrow in FIG. 14A) and resolution (darker curve) compared in averaged B-scans as a function of the mirror displacement range. The curves for the two measures cross at a displacement range of ˜0.3 μm, implying that an arrangement of mirror displacements derived from a distribution around 0.3 μm is the best choice for simultaneously preserving resolution and reducing speckle noise for this sample. For determining resolution only 20 frames were used to save time, since this number reduced speckle contrast by more than 80% by comparing with 100-frames averaged.

3. Defining an Optimum Subset of Deformable Mirror Configurations to Preserving the Resolution And Signal Intensity

While the above results show that an optimum mirror displacement range of ˜0.3 μm can be found (FIG. 13A-FIG. 14B), there is still considerable resolution loss, and substantial signal intensity loss (FIG. 13C). However, the huge number of potential DM configurations can make it is very inefficient to search for a global optimum DM configuration to mitigate the resolution and signal intensity loss. In analyzing the results, we found that the APM-OCT image intensity varied substantially between B-scans quite a lot (over 3 times, linear). Based on general principles, it could be expected that the brighter an individual image, the less distorted was the underlying PSF, suggesting that the subset of mirror displacement patterns yielding the brightest images might correspond to a set of minimally distorted PSFs.

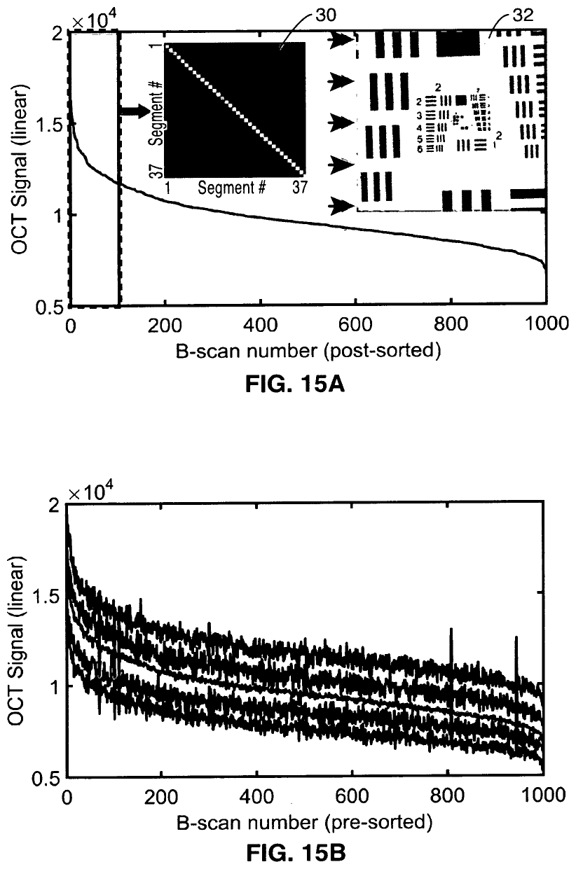

To examine this premise, an ensemble of 1000 B-scans was generated for a mirror displacement range of 0.3 μm and sorted them by their averaged signal intensities. FIG. 15A through FIG. 15 D illustrate that a subset of the mirror configurations reduces speckle while preserving resolution and signal strength.

FIG. 15A shows a plot of average intensity of 1000 APM-OCT B-scans plotted in descending order (the mirror displacement range was 0.3 μm), wherein the inset image 30 shows a covariance analysis of the top 100 mirror configurations, and the inset image 32 shows an enface test target image with grayscale coded arrows indicating the B-scan locations for the plots in FIG. 15B.

Referring to FIG. 15B, the same mirror configurations were applied across the ROI at the locations indicated in FIG. 15A. FIG. 15B shows APM-OCT signals from 1000 B-scans with the same DM configurations with that in FIG. 15 A. While average intensity varied somewhat for B-scans taken at different positions of the target (in FIG. 15B, arbitrary offsets were added for clarity purposes), the overall OCT signal plots were very similar, consistent with the idea that the shape of the plot was dictated by the PSFs corresponding to each mirror configuration, rather than by properties of the sample.

Next, a subset of first 100 mirror configurations corresponding to top 10% brightest images were selected for further examination. FIG. 15C shows a plot illustrating speckle contrast comparison for 100-frames-averaged APM-OCT images obtained with random and the selected “top 100” mirror configurations from the shaded region marked in FIG. 15A. We compared the ability of the selected top 10% subset of mirror configurations to reduce speckle noise with that of an equal number of random configurations for displacement ranges between 0 and 1.0 μm and found the selected subset of configurations performed almost as well.

Remarkably, however, the selected subset of configurations provided resolution up to ˜3-fold greater than the randomly generated configurations. FIG. 15D illustrates resolution plotted as a function of DM displacement range for different configurations: random (upper line), the top 10% (circles), or the top 2% (lower line). The left inset in FIG. 15D shows location on target grid for results plotted in right inset. The right inset in FIG. 15D show the vertically averaged cross-section OCT signal changes for different displacement ranges using the selected 2% configurations, showing there is a continuous contrast loss.

Even through the inset in FIG. 15D shows a continuous contrast loss, the resolution readout doesn't change. The resolutions achieved with the selected mirror configurations is always better than that with random configurations for displacement range greater than 0.25 μm and is asymptotically ˜3-fold better.

In conclusion, the “top 10%” subset of mirror configurations with displacement range of around 0.3 μm satisfies the triple constraints of greatly reducing speckle noise while simultaneously maximally preserving resolution and signal strength. More generally, the approach provides a rapidly implemented method for programming a deformable mirror to achieve these goals.

4. In Vivo Application of APM-AO-OCT Reduces Speckle Efficiently And Reveals Novel Structure

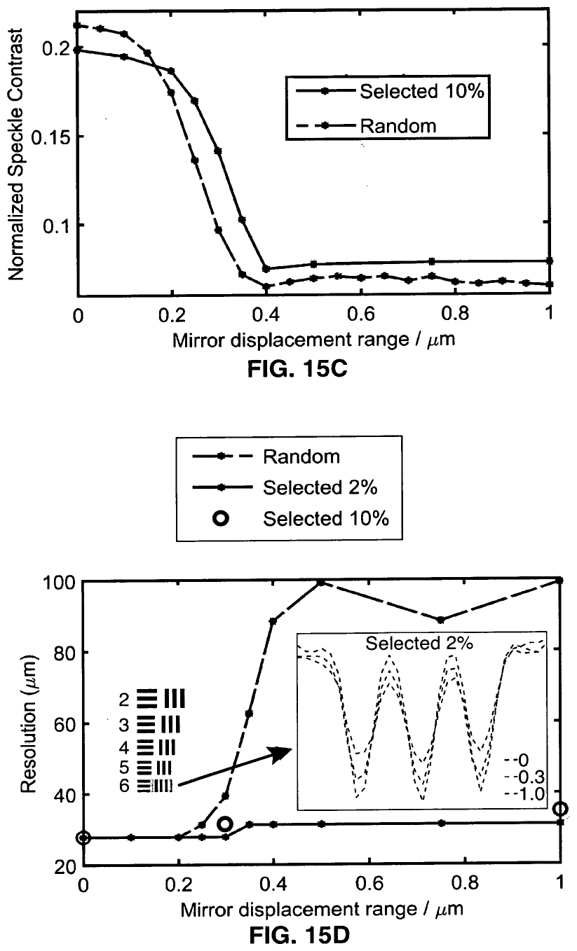

To examine the in vivo applicability of APM-AO-OCT the retinas of Balb/c mice were imaged using an interlaced B-scan acquisition protocol in which successive scans were acquired with or without APM. FIG. 16A—FIG. 16M illustrate a comparison of the efficiency of the averaging of APM-AO-OCT vs AO-OCT results in reducing speckle and revealing novel cellular structure in vivo. FIG. 16A-FIG. 16C are AO-OCT B-scans with N representing the number of images averaged. FIG. 16D-FIG. 16F are APM-AO-OCT B-scans with sample averaging corresponding to that used in panels FIG. 16A-FIG. 16C. The data in these panels were acquired with interlaced protocol. The focus of the AO-system was set to the IPL. The retinal layers are indicated in FIG. 16H, which is provided at the same scale as the OCT B-scans. FIG. 16G shows normalized speckle contrast of the IPL, for AO-OCT (rectangle in FIG. 16A; mostly upper line in FIG. 16G) and for APM-AO-OCT (rectangle in FIG. 16D; mostly lower line in FIG. 16G), plotted as function of the number of B-scan averaged. FIG. 16H shows a retinal plastic section of a C57131/6 mouse imaged with a 40× objective in a Nikon A1 microscope. FIG. 16I-FIG. 16L show averaged B-scans with the focus of the AO system shifted to the ONL; the shifted focus both increases the overall brightness of the images and narrows the width of the ONL scattering spots relative to those in FIG. 16A-FIG. 16F. FIG. 16M shows histology of the ONL from FIG. 16H presented with inverted contrast and magnified so as to have the same scale as panels FIG. 16I-FIG. 16L, and scale bar 50 μm. The arrow in FIG. 16L points to a periodic series of spots which is very similar to stacks of rod cell bodies in FIG. 16M. Abbreviations in FIG. 16H are as follows: NFL—nerve fiber layer, IPL—inner plexiform layer, INL—inner nuclear layer, OPL—outer plexiform layer, ONL—outer nuclear layer, ELM—external limiting membrane, BrM—Bruch's membrane.

Single B-scans exhibited substantial speckle that obscured even the highly scattered and extended structures, with little noticeable difference between scans taken with and without APM (FIG. 16A, FIG. 16D). The averages of 32 B-scans with and without APM had noticeably reduced levels of speckle (FIG. 16B, FIG. 16E). Notably, extended structures such as the ELM and Bruch's membrane appeared clearer in the image generated with APM. We quantified the speckle contrast in the region of the B-scans corresponding to the inner plexiform layer (IPL; dashed rectangles in FIG. 16A, FIG. 16D), as this region was bright, but showed no apparent structure. This quantification revealed that the averages of 32 scans taken with APM-AO-OCT had a reliably reduced level of speckle contrast relative to average of 32 scans taken with AO-OCT alone (FIG. 16B, FIG. 16G, arrow). The reduction in speckle contrast was evident for all sample sizes between 10 and 1000 (FIG. 16G). The AO-OCT results are consistent with previous observations showing that averaging per se leads to reduction in speckle contrast in in vivo imaging. This reduction was hypothesized to arise from the movements of subcellular organelles whose scattering gives rise to speckle. This hypothesis is supported by our observation that averaging of AO-OCT images of non-living targets does not per se much reduce speckle. Nevertheless, APM-AO-OCT more efficiently reduces speckle. Thus, the average of 32 scans with APM-AO-OCT (FIG. 16E) appears comparable to that of 1000 scans taken with AO-OCT alone (FIG. 16C).

In addition to its greater efficiency than pure averaging in reducing speckle noise, APM-AO-OCT also serves to increase the confidence with which the experimenter can draw conclusions about structures. To illustrate this point, we compare OCT images taken with the two methods after shifting the focus of the AO-system to the ONL FIG. 16I-FIG. 16L. The ONL comprises the cell bodies of the photoreceptors, which are developmentally arranged in vertical stacks of 10-11 (FIG. 16G, histology). The average of 32 AO-OCT scans (FIG. 16I) shows spots of increased scattering that might be hypothesized to arise from the photoreceptor nuclei. However, the speckle noise is such that the hypothesis is dubitable. The average of 32 APM-AO-OCT B-scans strengthens the hypothesis (FIG. 16J). The comparison of averages of 1000 B-scans (FIG. 16J-FIG. 16K) leads to even greater conviction that the bright spots arise from rod nuclei: thus, for example, in FIG. 16L one can observe a number of rows of such spots which have the same vertical spacing and in some cases the expected total number as rod nuclei seen in ONL histology (FIG. 16M; contrast-inverted from FIG. 16H). While the hypothesis that photoreceptor nuclei can be visualized with APM-AO-OCT (and to a lesser extent, AO-OCT) needs to be tested further, the evidence from the vertical and lateral spacing as well as size is substantial, and demonstrates the potential for APM-AO-OCT for producing novel discovery. Thus, for example, it is possible that the variation in the brightness of the ONL spots reflects diurnally or otherwise changing structural and/or functional properties of the cell bodies and nuclei.

To explore the full potential of APM-AO-OCT to reduce speckle and uncover structure in vivo, we applied the method to volumetric data acquisition, arranging the focus of the AO-system to be at the uppermost retinal layers, and comparing AO-OCT with APM-AO-OCT as before.

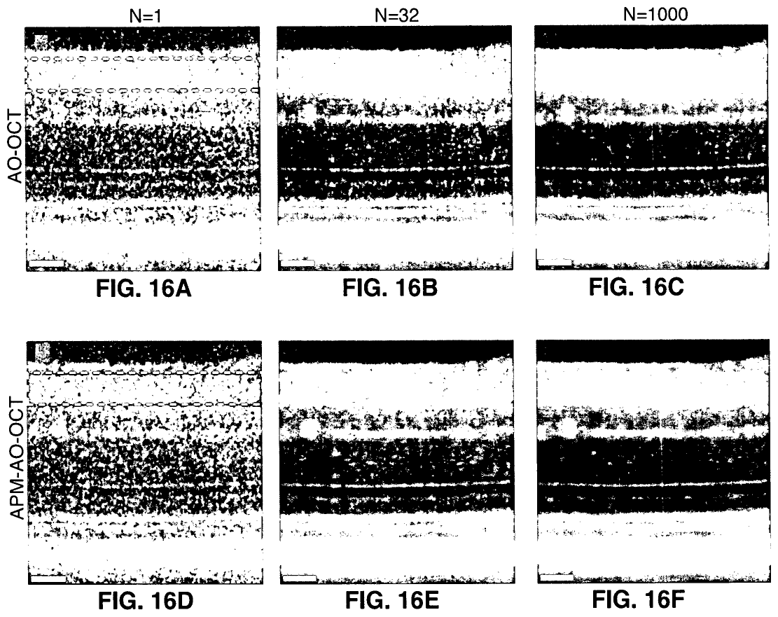

FIG. 17A-FIG. 17J illustrate visualization of cellular scale structures in retinal layers with in vivo volumetric APM-AO-OCT. FIG. 17A is a B-scan from a 560×280×320 μm³ retinal volume imaged 50 times with interlaced AO-OCT and APM-AO-OCT, aligned and averaged; the AO system was optimized for focus on the outer retina. The dashed lines indicate planes at which enface images were extracted for FIG. 17A-FIG. 17J respectively. FIG. 17B-FIG. 17C show enface presentation of a 0.85 μm digital section at the depth locus indicated by red dashed line in a for AO-OCT (FIG. 17B) and APM-AO-OCT (FIG. 17C) respectively. White arrows point to thin line structures that can be excluded as being blood vessels, and likely represent the outermost ganglion cell axons. FIG. 17D-FIG. 17E show enface presentation of a 0.85 μm digital section at the depth locus indicated by green dashed line in a, 10 μm deeper into the retina than FIG. 17B-FIG. 17C. Magnified presentations reveal relatively brighter (gray) contiguous regions with especially bright dots enclosed; these regions are hypothesized to reveal displaced amacrine cells, which are known to reside in this layer. FIG. 17F shows electron microscopic image of an amacrine cell image (from 45). FIG. 17G-FIG. 17H show enface presentations of 0.85 μm digital sections for AO-OCT and APM-AO-OCT with focus on the NFL. Speckle noise reduction by APM-AO-OCT enables more confident discrimination between blood vessels and axon fiber bundles, with interlaced protocol. FIG. 17I-FIG. 17J show enface OCT angiography (phase-variance analysis) with AO-OCT (FIG. 17I) and APM-AO-OCT (FIG. 17J). The aperture phase modulation substantially reduces the phase-variance signal in the APM-AO-OCT data, while the interlaced AO-OCT data preserves the signal. The scale bar is 100 μm (white) for all panel except FIG. 17F, where it represents 1 μm. Abbreviations in FIG. 17A are as follows: NFL—nerve fiber layer, OPL—outer plexiform layer, ELM—external limiting membrane, RPE—retinal pigment epithelium.

Enface presentation of single averaged volume layer showed an enhanced reduction of speckle by APM-AO-OCT (FIG. 17B vs. FIG. 17C) and several linear structures (arrows) not discernible in the corresponding AO-OCT image. The averaged single layer about 10 μm deeper in the retina (FIG. 17D, FIG. 17E) revealed several regions with intensity greater than the surround which include bright spots. Based on a comparison with published histology (FIG. 17F, low-power electron microscopy), we hypothesize that these regions represent displaced amacrine cells. Another comparison at the level of the NFL is provided FIG. 17G, FIG. 17H. Here APM-AO-OCT provides a greater reduction of speckle noise and improved confidence in the discrimination of blood vessels from ganglion cells axon fiber bundles. A potential downside of APM-AO-OCT is that its utility for OCT angiography is reduced (FIG. 17I, FIG. 17J). However, this problem can be dealt with the interlaced scanning protocol, as the AO-OCT-alone scans retain the angiographic information (FIG. 17I). Furthermore, the comparison of the averages from the interlaced protocol may lead to insight into the scattering structures seen with the AO-OCT images (compare FIG. FIG. 16K, FIG. 16L).

D. Exemplary Methods

The following description provides eight examples for computer-implementation of above-described technology, which may be implemented as machine-readable instructions or code in application software 36 for operating the DM 12a and/or performing various data acquisition and image processing techniques detailed executing. While the following examples are directed to phase modulation using a deformable mirror, it is appreciated that each of the methods may equally be employed with use of any number of phase modulation optics (e.g. SLM, LDL, etc.)

### Example 1 (Data Acquisition)

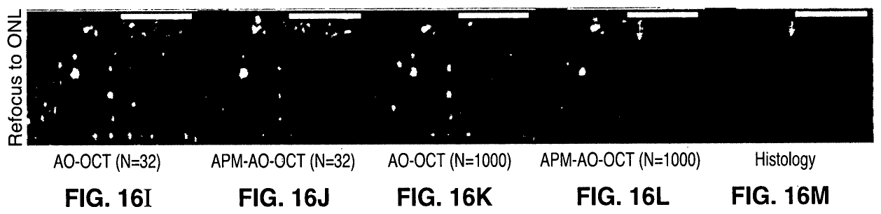

FIG. 19 shows a flow diagram for a method 100 that can be employed for data acquisition comprising the following steps:

1. System initialization at step 102, e.g. power on, parameters loading and setting, memory space allocation, etc.;

2. Sample alignment with normal OCT operation and optimize the image using the wavefront sensor-less AO-OCT method to get the brightest image at step 104;

3. Change and record the corresponding mirror shape at step 106;

4. Run 1000 trial of random aperture phase modulation (scan) and record the images and their corresponding mirror configurations for brightest image selection at step 108 (this operation loops back to step 106 until N=No;

5. Select the top M % images with the brightness in the region of interest as the metric, sort the images by intensity descending order and record the corresponding deformable mirror configurations at step 112;

6. APM Data acquisition is then performed with selected mirror configurations:

a. Load the top M % mirror configurations from step 112;

b. Change the DM shape using one mirror configurations from the loaded set at step 114;

c. Acquire a single OCT B-scan and save the raw OCT spectrum data at step 118;

d. Repeat steps 114 and 118 at step 120 until N=N₁ times;

e. Flatten the DM shape using zero or the mirror shape optimized by

AO at step 122;

f. Acquire a single OCT B-scan and save the raw OCT spectrum data at step 124;

g. Repeat step 122 and step 124 at step 126 until N=N₁ times;

h. Move the Y-scanner one step at step 116, and repeat step 114 through step 126 until N=N₂ times;

7. End and stop data acquisition automatically at step 128.

In one exemplary configuration, MATLAB—LabVIEW mix programming may be implemented for use in data acquisition. The function of this code is to control the DM. Code may be implemented to control the DM via MATLAB-LabVIEW mix programming technique using the following inputs: Amp_Array: control random amplitude array, Mirror_control: control mode, MirrorShape: pre-set mirror shape mode, error in, Simulate?: is it running as a simulation (no hardware involved) and a few outputs: Mirror configurations, init_mirror_pos: initial mirror position (check point), Real_Pos: readout position after sending Amp_Array to the DM, Saturated_Seg: output the marks for each saturated segment, error out

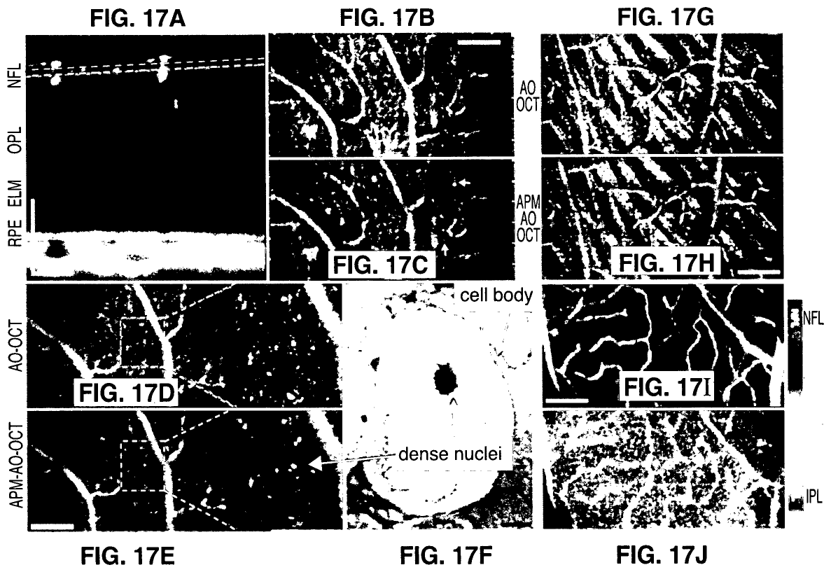

FIG. 20 shows an exemplary method 150 that may be employed for

OCT data processing and post-processing comprising the following steps:

1. Input in the raw OCT spectrum data in a B-scan at step 152;

2. DC subtraction (average the whole B-scan spectrum as the DC, then subtract it from the B-scan spectrum) at step 154;

3. Use the calibrated OCT spectrometer parameters to interpolate the OCT B-scan spectrum x coordinate from pixel space to wavelength space, then further to frequency space (k space, 1/wavelength) at step 156;

4. Dispersion compensation of the k-space phase up to third order polynomial at step 158;

5. Zero padding, then Fast Fourier Transform (FFT) of the k-space data at step 160;

6. Post-FFT processing, including linear or log display, casting the image into different display range; intensity calculation of the ROI; registration; averaging, etc. at step 162.

This is an exemplary standard Fourier/Spectral domain OCT process procedure, which may be applied to both real-time/postprocessing.

### Example 2

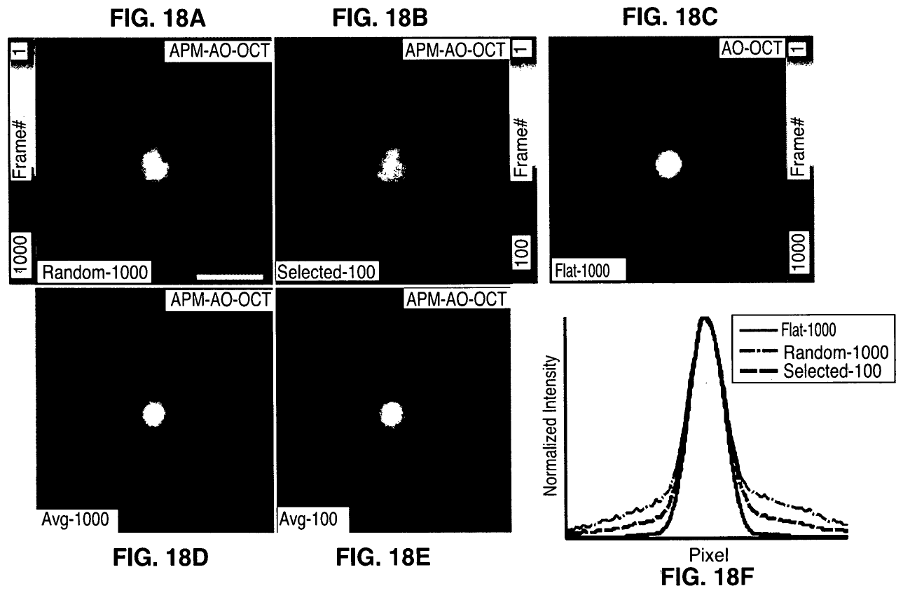

One aspect of the technology is a method for generating a set of “random” psf by APM that can be used to search for optimum psf's. FIG. 21 shows an exemplary method 170 that may be employed to generate a set of “random” mirror configurations which can be used for further optimization of OCT signal. In the example shown, the method comprises the following steps:

1. Providing the DM in a flat zero/optimized configuration at step 172; and 2. Adding random phase displacement for each segment and recording the corresponding image and mirror configurations at step 174.

Steps 172 and 174 are repeated until all presets are applied.

LabVIEW programming may also be implemented to generate randomizing for different mirror segments, including saving mirror configurations. In such implementation, the selected mirror configurations are loaded as input if work in “loaded” mode, and the implementation outputs the mirror configurations (either random or loaded/selected).

### Example 3

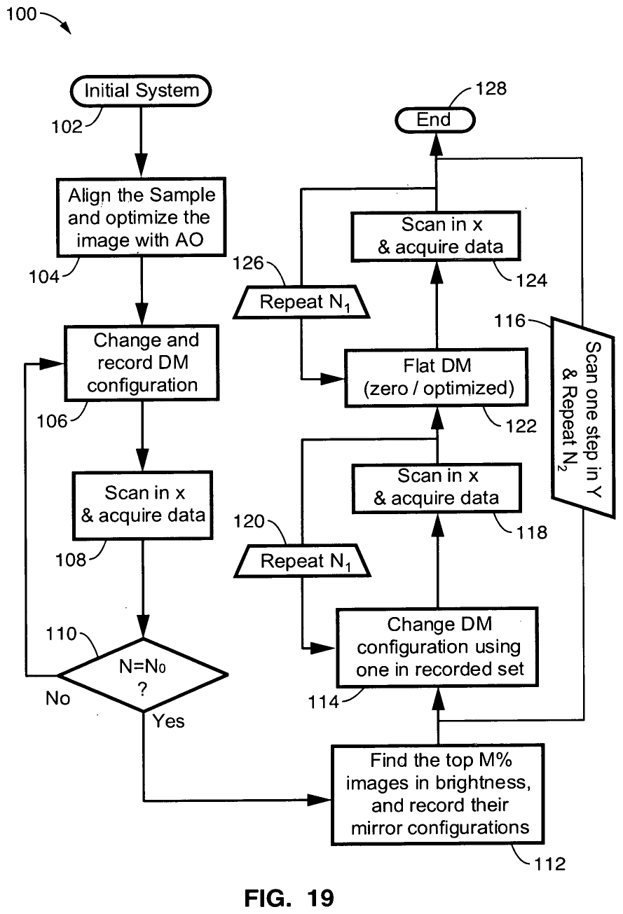

FIG. 22 shows an exemplary method 180 that may be employed for searching for optimum sets of PSFs for APM-AO-OCT for a given sample comprising the following steps:

1. Calculating the image brightness for all the images recorded in method 170 at step 180;

2. Sort the image by intensity descending order and record the corresponding deformable mirror configurations at step 184; and

3. Select the mirror configurations corresponding to the top M % brightest images at step 184.

Matlab can be used to find the top, e.g. 10%, mirror configurations corresponding to brightest images. The following inputs would be employed: Linear_Amp_FFT2X.tif: Certain number, e.g. 1000, B-scans with random mirror configurations, random_z: the corresponding random mirror configurations, opt: option, interlaced scan mode, ROI: region of interest for calculation the image intensity, and a few outputs: Mirror configurations, Max_random_100: the top 10% mirror configurations with brightest images, Max_random_1000_7: the top 10% with certain interlaced scan mode for loading to the LabVIEW code.

Exemplary code to find the top mirror configurations corresponding to brightest images is provided in Table 2.

### Example 4

Another aspect of the technology is a method to acquire interlaced B-scan with AO-OCT and APM-AO-OCT B-scans. This method provides for acquisition of standard and speckle free images for further image processing. The method also provides for:

a. Acquisition of intrinsic sample motion to compare to speckle free APM-AO-OCT imaging.

b. Comparison between static and dynamic structures between biological sample.

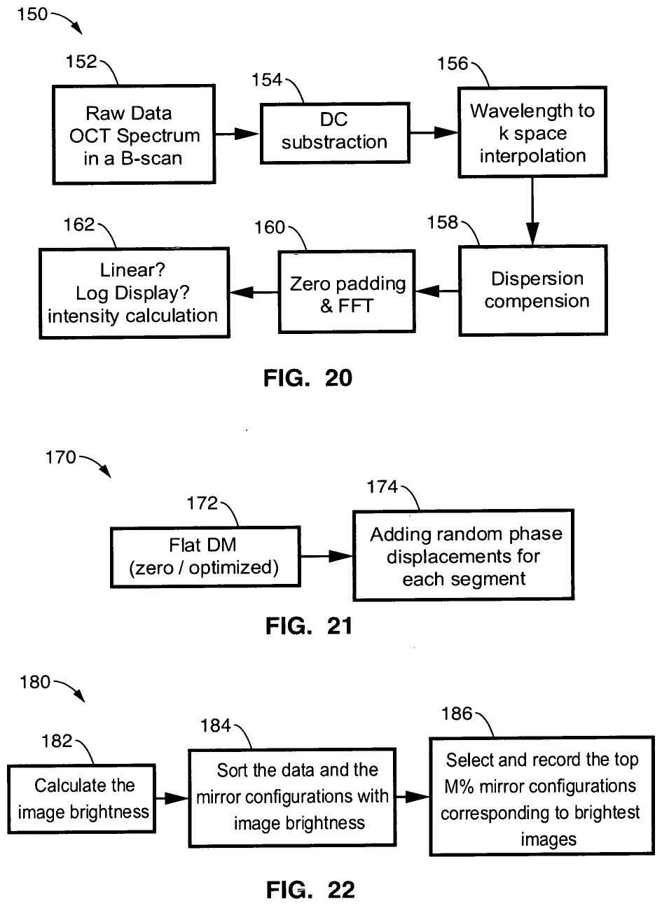

FIG. 23 shows an exemplary method 200 that may be employed to acquire interlaced B-scan with AO-OCT and APM-AO-OCT B-scans, comprising the following steps:

1. Load the top M % mirror configurations and change the DM shape using one mirror configurations from the loaded set at step 202;

2. Acquire a single OCT B-scan and save the raw OCT spectrum data at step 204;

3. Repeat steps 202 and 204 N₁ times at step 208;

4. Flatten the DM shape using zero or the mirror shape optimized by AO at step 210;

5. Acquire a single OCT B-scan and save the raw OCT spectrum data at step 212;

6. Repeat step 210 and 212 N₁ times at step 214;

7. Keep the Y-scanner zero (doesn't move), repeat step 202 to 214 N₂ times at step 206.

### Example 5

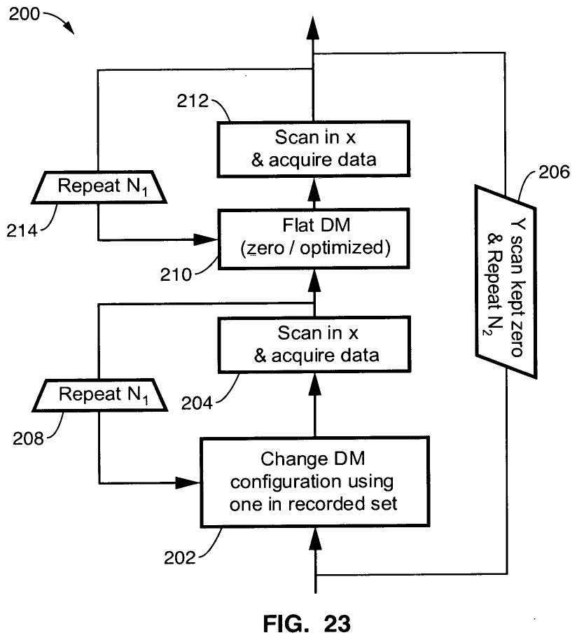

Another aspect of the technology is a method to extend APM-AO-OCT interlaced B-scan acquisition to volumetric data acquisition by acquiring Serial B-scans and build OCT volume from that (slow data acquisition or static sample). FIG. 24 shows an exemplary method 220 to extend the interlaced B-scan data acquisition method with AO-OCT and APM-AO-OCT to allow acquisition of standard and speckle free volumes for further processing and comparison, comprising the following steps:

1. Load the top M % mirror configurations and change the DM shape using one mirror configurations from the loaded set at step 222;

2. Acquire a single OCT B-scan and save the raw OCT spectrum data at step 224;

3. Repeat step 222 and step 224 N₁ times at step 228;

4. Flatten the DM shape using zero or the mirror shape optimized by AO at step 230;

5. Acquire a single OCT B-scan and save the raw OCT spectrum data at step 232;

6. Repeat step 230 and 232 N₁ times at step 234;

7. Move the Y-scanner position by one step and repeat step 222-step 234 N₂ times until the entire FOV (field-of-view) was scanned at step 226.

### Example 6

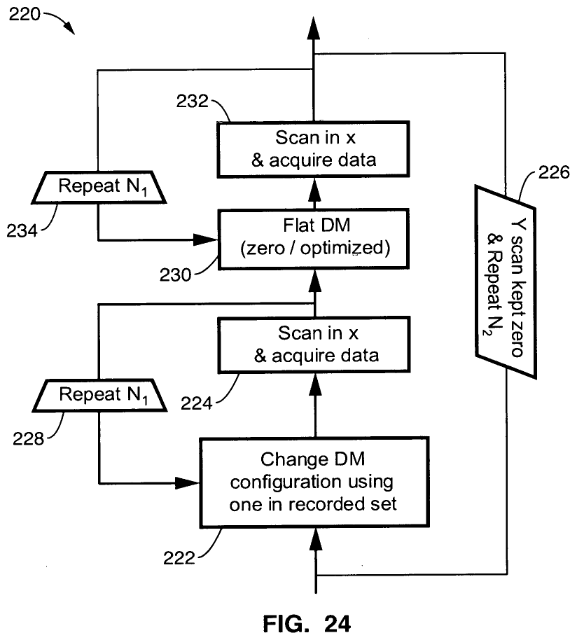

The technology also includes a method to extend APM-AO-OCT interlaced B-scan acquisition to volumetric data acquisition by acquiring Serial Volumes and build APM-AO-OCT interlaced volume from that (fast data acquisition or moving sample). FIG. 25 shows an exemplary method 250 to extend the interlaced B-scan data acquisition method with AO-OCT and APM-AO-OCT to allow interlaced volume acquisition of standard and speckle free for further processing and comparison comprising the following steps:

1. Load the top M % mirror configurations and change the DM shape using one mirror configurations from the loaded set at step 252;

2. Acquire a single OCT volume scan and save the raw OCT spectrum data at step 254;

3. Flatten the DM shape using zero or the mirror shape optimized by AO at step 256;

4. Acquire a single OCT volume and save the spectrum data at step 258;

6. Repeat step 252 to step 258 N₁ times at step 260.

### Example 7

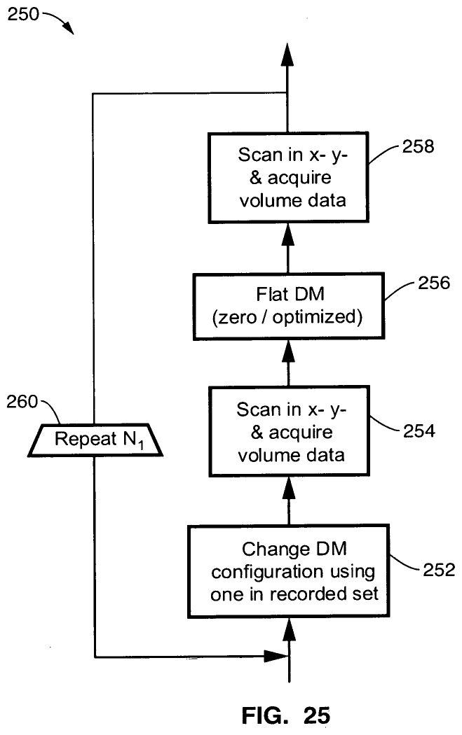

A method to deform segmented wavefront correctors that allows maintained lateral resolution while varying PSF is also included in the technology. FIG. 26 shows an exemplary method 270 to further analyze the selected mirror configurations to create mirror patterns that allows maintaining lateral resolution while varying PSF comprising the following steps:

1. Calculating the image brightness for all the images recorded in method 270 at step 272;

2. Sort the image data and their corresponding mirror configurations by intensity descending order at step 274;

3. Select the mirror configurations corresponding to the top M % brightest images at step 276;

4. Perform histogram analysis ring by ring according to the distance from mirror segments to the DM center at step 278;

5. Output from the histogram at 280 the outer segments that have bigger randomization amplitude than the more central ones, which suggests a potential way to further optimize the mirror configurations.

Matlab can be used to test the optimal mirror configuration histogram ring by ring with the following inputs: random_z: random mirror configurations, Sorted_random_z: sorted random mirror configurations, and output: Mirror configurations.

Table 3 provides code used to test the optimal mirror configuration histogram ring by ring.

### Example 8

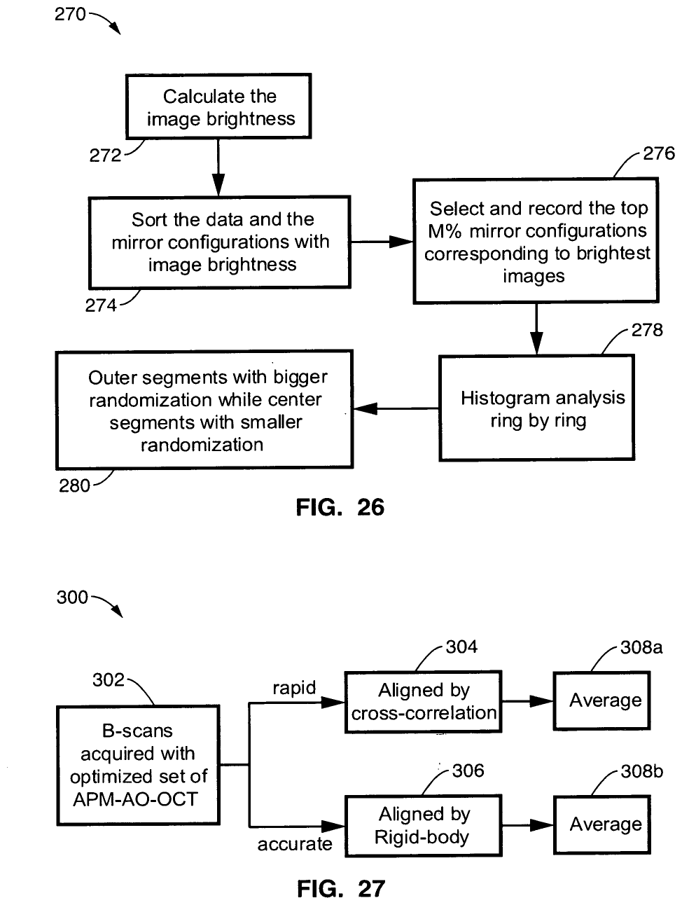

The technology of the present description also reduces speckle by averaging optimized set of APM-AO-OCT B-scans. FIG. 27 shows an exemplary method 300 to average optimal set of APM-AO-OCT B-scans to suppress the speckle comprising the following steps:

1. acquire APM-AO-OCT B-scans at step 302; and

2a. align with cross-correlation at step 304 for rapid averaging at step 308a to suppress the speckle; or

2b. align with Rigid-body transformation at step 306 for accurate averaging at step 308b to suppress the speckle.

F. Summary and Discussion

Adaptive optics has revolutionized image science by enabling image systems to perform at their diffraction limits, and thereby reveal a wealth of novel structure. AO systems operate by actively controlling the wavefront at the system pupil aperture and have been implemented in imaging systems for in vivo ophthalmic imaging, including Scanning Laser Ophthalmoscopy (SLO) and OCT systems. OCT imaging systems employ partially coherent light sources to extract depth scattering profiles of tissue, and as with all systems that use such sources, are subject to speckle noise, which substantially reduces their signal-to-noise ratio. The systems and methods presented herein provide a novel approach to speckle noise reduction in OCT. This approach exploits small scan-to-scan modulations of the phase at the aperture of an AO-OCT system produced by sub-micron displacements of the segments of a deformable mirror (FIG. 1). We established that an optimum mirror displacement range can be found which simultaneously greatly reduces speckle noise and maintains image resolution (FIG. 13A-FIG. 14B), and that a small subset of the mirror configurations can further improve resolution and preserve signal intensity (FIG. 15A-FIG. 15D). Finally, we demonstrated APM-AO-OCT can be used in vivo to efficiently reduce speckle noise and discover novel structures (FIG. 16A-FIG. 17J).

1. Mechanism of APM-AO-OCT: Perturbations of The System Point-Spread Function/Active PSF Shaping

In an OCT system, the point-spread function (PSF) is defined axially by the source coherence length and determined laterally by the NA of the system aperture (Methods). Because the sampling unit in OCT is the A-scan, the lateral extent of the PSF varies with depth, achieving its NA-limited minimum at the focal depth, which is the diffraction limit in AO-OCT approaches. Aperture phase modulation (APM) necessarily perturbs the OCT PSF shape, but primarily affects its x-, y-distribution.

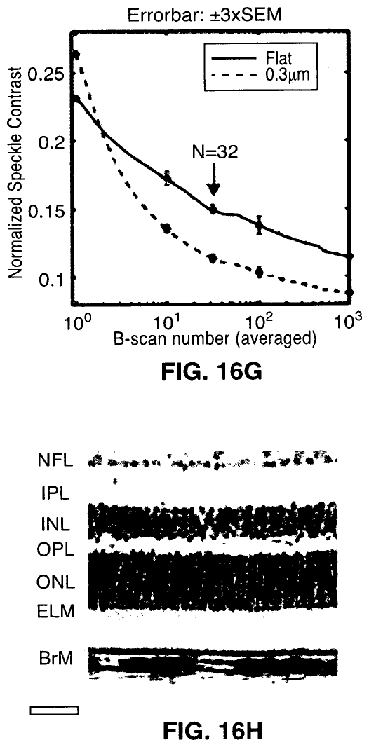

The effects of APM on the PSF can be visualized by focusing the OCT beam onto a CMOS camera. FIG. 18A-FIG. 18F show a comparison of the lateral extent of AO-OCT and APM-AO-OCT PSFs at the focus. All PSF images were obtained by focusing the beam onto a CMOS camera.

Note that, these images represent the “1-way” or incoming PSF of the system, whereas in application the effective PSF results from two passes through the system aperture.

Each of a series of 1000 APM-AO-OCT PSFs exhibit a central power density with random extensions of lower power (FIG. 18A), while a similar sample of 1000 AO-OCT PSFs are identical (FIG. 18C). The “top 100” with brightest maximum intensity of the APM-AO-OCT sample is more compact (FIG. 18B), as further emphasized by comparison of the averages (FIG. 18C, FIG. 18D), and comparison of line scans through the averaged PSF centers (FIG. 18F). This analysis provides support for the premise that the averaging of scans taken with APM-AO-OCT efficiently reduces speckle contrast because the randomly distorted PSFs encompass different sets and numbers of scatterers, while the maintained centroid of the PSFs captures information from larger scale structural elements in the sample.

Further implementation may include the class of mirror displacement configurations that minimize speckle contrast while maintaining resolution and image brightness that are modeled by additional characterization of the mirror configurations that give optimum performance, and by theoretical analysis of the corresponding perturbed wavefronts. Thus, for example, histogram analysis of the DM segment displacements of the “top 100” configurations as a function of distance from the DM pupil center revealed that the outermost actuators underwent uniform variation over the full range of deformation, while the inner actuator displacements followed a Gaussian distribution with a restricted range. Thus, configurations characterized by the subset of Zernike aberrations of the class Zⱼ^{±j} may be especially important in optimizing APM-AO-OCT.

2. Comparison with Similar Methods of Speckle Reduction

Many different approaches to reducing speckle noise in imaging systems employing coherent light have been proposed. Previously proposed methods that vary the properties of the incoming beam suffer from being uncontrollable: first, the wavefront distribution is not under precise experimenter control, thus the distorted PSF is not readily obtained, losing the capability for optimizing; second, the class of permitted distortions is limited and cannot be easily and precisely changed, thus, is not a universal way for different systems with variable wavelength and/or samples. APM-AO-OCT overcomes these deficits and gives the experimenter precision control on a very rapid trial-by-trial basis, providing a quantified and repeatable way to further explore and optimize the method. Furthermore, APM-AO-OCT is intrinsically compatible with adaptive optics, offering a great chance to pursue high resolution imaging, especially for in vivo applications.

Recently, it was also found that averaging of multiple, precisely aligned volumes for in vivo OCT imaging could reduce speckle noise and reveal novel cellular scale structure. It was hypothesized that such averaging is effective because of the random movement of sub-PSF size scattering elements in cells. This approach is passive, however, and limited by the time scale and extent of the underlying scatterer motions, which requires certain time to decorrelate the speckle pattern between images. The in vivo results presented here confirm the effectiveness of pure averaging, but also show that the active approach of APM-AO-OCT can be considerably more efficient.

3. Additional Considerations/Applications

It is appreciated that the APM-AO-OCT techniques are not limited to the systems and methods disclosed herein. Because APM-OCT suppresses the speckle and makes the image smoother, it improves the intensity-based peak location detection of the structures in the retina, which for example, may be harnessed to provide more precise retinal optophysiology signal measurements in length changes, as well better OCT imaging for high scattering tissue, such as brain imaging.

While the systems and methods of the present disclosure focus on creating random phase changes inside the pupil aperture, other ways are contemplated. Other PSF shaping methods need may be used to find the further optimal ways to suppress the speckle while maintain the resolution, contrast and/or intensity.

Finally, the broad adoption of adaptive optics continues to revolutionize imaging science and has spurred the development of wavefront corrector with increasing numbers of segments and speed. In principle, APM-AO-OCT could also be implemented by using spatial light modulators (SLM), digital micro-mirror devices (DMD), or other deformable mirrors (e.g. AlpAO, BMC—Boston micromachines, Inc. etc.). Also, OCT systems capable of megahertz A-scan rates have been developed. The marriage of these two advancing technologies may enable routine implementation of APM-AO-OCT in clinical and research settings to assure clinic diagnose and basic science discovery.

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general-purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure (s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, that the terms processor, hardware processor, computer processor, central processing unit (CPU), and computer are used synonymously to denote a device capable of executing the instructions and communicating with input/output interfaces and/or peripheral devices, and that the terms processor, hardware processor, computer processor, CPU, and computer are intended to encompass single or multiple devices, single core and multicore devices, and variations thereof.

From the description herein, it will be appreciated that the present disclosure encompasses multiple embodiments which include, but are not limited to, the following:

A method of speckle free optical coherence tomographic imaging, the method comprising: (a) providing a confocal coherent detection system with an entrance aperture; (b) controlling modulation of entrance aperture aberrations; (c) generating a set of optimally aberrated point-spread functions (PSFs) in a sample; and (d) producing an image of the sample using the generated optimally aberrated point-spread functions.

The method or system of any preceding or subsequent embodiment, wherein said modulation of entrance aperture aberrations is controlled with adaptive optics elements selected from the group of elements consisting of a deformable mirror, a segmented mirror and a spatial light modulator.

The method or system of any preceding or subsequent embodiment, wherein generation of said set of optimally aberrated point-spread functions (PSFs) in a sample comprises: (a) modulating a phase inside the imaging system pupil aperture with a segmented deformable mirror to produce minor perturbations in the point spread function (PSF) and create un-correlated speckle patterns between B-scans; (b) applying an averaging technique to the patterns to wash out speckle but maintain structures; and (c) searching for optimally aberrated point-spread functions.

The method or system of any preceding or subsequent embodiment, further comprising: (a) acquiring an interlaced B-scan, an adaptive optics-optical coherence tomography (AO-OCT) scan, and an aperture phase modulation-adaptive optics-optical coherence tomography (APM-AO-OCT) B-scan; and (b) producing standard and speckle free images from said scans.

The method or system of any preceding or subsequent embodiment, further comprising: (a) acquiring speckle free aperture phase modulation-adaptive optics-optical coherence tomography (APM-AO-OCT) images of a biological sample; (b) acquiring intrinsic sample motion images; (c) comparing intrinsic sample motion images and speckle free APM-AO-OCT images; and (d) identifying static and dynamic structures of the biological sample.

The method or system of any preceding or subsequent embodiment, further comprising: (a) acquiring serial aperture phase modulation-adaptive optics-optical coherence tomography (APM-AO-OCT) interlaced B-scans of a sample; and (b) building an optical coherence tomography (OCT) volume of the sample from the APM-AO-OCT interlaced B-scans of the sample with slow data acquisition or static sample scans.

The method or system of any preceding or subsequent embodiment, further comprising: (a) acquiring serial volumetric aperture phase modulation-adaptive optics-optical coherence tomography (APM-AO-OCT) interlaced B-scans of a sample; and (b) building APM-AO-OCT interlaced volume of the sample from the volumetric APM-AO-OCT interlaced B-scans of the sample with fast data acquisition or moving sample scans.

A method of speckle free optical coherence tomographic imaging, the method comprising: (a) providing an optical coherence tomographic system with segmented wavefront correctors; (b) deforming the segmented wavefront correctors to maintain lateral resolution while varying point-spread functions (PSFs); and (c) producing an image of a sample using generated optimum point-spread functions.

The method or system of any preceding or subsequent embodiment, further comprising: (a) randomly deforming the segmented wavefront correctors to produce uncorrelated speckle patterns; (b) searching for optimum point-spread functions; and (c) averaging optimized sets of aperture phase modulation-adaptive optics-optical coherence tomography (APM-AO-OCT) B-scans.

The method or system of any preceding or subsequent embodiment, further comprising: (a) optimizing a mirror segment displacement range; and (b) selecting a subset of mirror configurations within the optimum range to satisfy triple constraints of greatly reducing speckle noise while simultaneously maximally preserving resolution and signal strength.

A method for generating a set of random mirror configurations for use in optimization of an aperture phase modulation-adaptive optics-optical coherence tomography (APM-AO-OCT) signal where a deformable mirror (DM) having mirror segments is used, the method comprising: (a) performing an optical coherence tomography (OCT) scan and acquiring an image; (b) adding random phase displacement for each mirror segment; (c) recording image and mirror configurations corresponding to the phase displacement; and (d) repeating steps (b) and (c) until a set of presets are exhausted.

A method for searching for optimum sets of point spread functions (PSFs) for aperture phase modulation-adaptive optics-optical coherence tomography (APM-AO-OCT) for a given sample where a deformable mirror (DM) having mirror segments is used, the method comprising: (a) performing an optical coherence tomography (OCT) scan and acquiring an image; (b) adding random phase displacement for each mirror segment; (c) recording image and mirror configurations corresponding to the phase displacement; (d) calculating image brightness for all recorded images; (e) sorting images by brightness and recording corresponding mirror configurations; and (f) selecting mirror configurations corresponding to a selected percentage of highest image brightness.

A method for acquiring interlaced B-scans with adaptive optics-optical coherence tomography (AO-OCT) and aperture phase modulation-adaptive optics-optical coherence tomography (APM-AO-OCT) B-scans for acquiring standard and speckle free images where a deformable mirror (DM) having mirror segments is used, the method comprising: (a) performing an optical coherence tomography (OCT) X-direction scan and acquiring an image; (b) adding random phase displacement for each mirror segment; (c) recording image and mirror configurations corresponding to phase displacement; (d) calculating image brightness for all recorded images; (e) sorting images by brightness and recording corresponding mirror configurations; (f) loading mirror configurations corresponding to a selected percentage of highest image brightness; (g) changing shape of the DM using one mirror configuration from the loaded mirror configurations; (h) acquiring a single OCT B-scan and saving raw OCT spectrum data; (i) repeating steps (g) and (h) N1 times; (j) flattening the DM shape using zero or mirror shape optimized by adaptive optics (AO); (k) acquiring a single OCT B-scan and saving raw OCT spectrum data; (l) repeating steps (j) and (k) N1 times; and (m) without changing Y-direction scan position, repeating steps (g) through (l) N2 times.

A method for extending interlaced B-scan data acquisition with adaptive optics-optical coherence tomography (AO-OCT) and aperture phase modulation-adaptive optics-optical coherence tomography (APM-AO-OCT) to allow acquisition of standard and speckle free volumes where a deformable mirror (DM) having mirror segments is used, the method comprising: (a) performing an optical coherence tomography (OCT) X-direction scan and acquiring an image; (b) adding random phase displacement for each mirror segment; (c) recording image and mirror configurations corresponding to phase displacement; (d) calculating image brightness for all recorded images; (e) sorting images by brightness and recording corresponding mirror configurations; (f) loading mirror configurations corresponding to a selected percentage of highest image brightness; (g) changing shape of the DM using one mirror configuration from the loaded mirror configurations; (h) acquiring a single OCT B-scan and saving raw OCT spectrum data; (i) repeating steps (g) and (h) N1 times; (j) flattening the DM shape using zero or mirror shape optimized by adaptive optics (AO); (k) acquiring a single OCT B-scan and saving raw OCT spectrum data; (l) repeating steps (j) and (k) N1 times; and (m) changing Y-direction scan position by one step and repeating steps (g) through (l) N2 times until entire field-of-view (FOV) is scanned.

A method for extending interlaced B-scan data acquisition with adaptive optics-optical coherence tomography (AO-OCT) and aperture phase modulation-adaptive optics-optical coherence tomography (APM-AO-OCT) to allow interlaced volume acquisition of standard and speckle free volumes where a deformable mirror (DM) having mirror segments is used, the method comprising: (a) performing an optical coherence tomography (OCT) X-Y direction scan and acquiring an image; (b) adding random phase displacement for each mirror segment; (c) recording image and mirror configurations corresponding to phase displacement; (d) calculating image brightness for all recorded images; (e) sorting images by brightness and recording corresponding mirror configurations; (f) loading mirror configurations corresponding to a selected percentage of highest image brightness; (g) changing shape of the DM using one mirror configuration from the loaded mirror configurations; (h) acquiring a single OCT volume scan and saving raw OCT spectrum data; (i) flattening the DM shape using zero or the mirror shape optimized by adaptive optics (AO); (j) acquiring a single OCT volume and saving the spectrum data; and (k) repeating steps (g) through (j) N1 times.

A method for creating mirror patterns that allows maintaining lateral resolution while varying point-spread function (PSF) where a deformable mirror (DM) having mirror segments is used, the method comprising: (a) performing an optical coherence tomography (OCT) scan and acquiring an image; (b) adding random phase displacement for each mirror segment; (c) recording image and mirror configurations corresponding to the phase displacement; (d) calculating image brightness for all recorded images; (e) sorting images by brightness and recording corresponding mirror configurations; (f) selecting mirror configurations corresponding to a selected percentage of highest image brightness; and (g) performing histogram analysis ring by ring according to distance from mirror segments to the center of the deformable mirror.

The method or system of any preceding or subsequent embodiment, wherein outer segments have greater randomization than more central segments.

A method for averaging an optimized set of aperture phase modulation-adaptive optics-optical coherence tomography (APM-AO-OCT) B-scans to suppress the speckle comprising the following steps: rapidly averaging the B-scans by aligning the B-scans with cross-correlation; and accurately averaging the B-scans by aligning the B-scans with rigid-body transformation.

A system for performing aperture phase modulation (APM) with adaptive optics (AO) for speckle reduction and structure extraction in optical coherence tomography (OCT), comprising: (a) a phase modulating element having a surface for receiving a beam of light, said beam of light directed at a system aperture; (b) a processor coupled to the phase modulating element; and (c) a non-transitory memory storing instructions executable by the processor; (d) wherein said instructions, when executed by the processor, perform steps comprising: (i) controlling the phase modulating element to randomize light modifying properties across a plurality of regions on the surface and generate a first random phase variation pattern across the system aperture; (ii) performing a first B-scan based on the first random phase variation pattern; (iii) controlling the phase modulating element to randomize light modifying properties across a plurality of regions and generate a second random phase variation pattern across the system aperture; (iv) performing a second B-scan based on the second random phase variation pattern; (v) wherein successive scans produce minor perturbations in a point spread function (PSF) associate with the beam and create un-correlated speckle patterns between B-scans.

The method or system of any preceding or subsequent embodiment, wherein the phase modulating element defines a wavefront across the system aperture to correct aberrations resulting in a compact PSF.

The method or system of any preceding or subsequent embodiment, wherein the instructions are further configured for: alternating between an optimum AO configuration and a randomized configuration modulated from the optimum AO configuration between successive B-scan phases.

The method or system of any preceding or subsequent embodiment:

wherein the phase modulating element comprises a segmented deformable mirror having a plurality of segments corresponding to each of the surface regions, each of the segments being independently controllable by the processor to independently control a displacement of the plurality of segments to randomize said surface.

The method or system of any preceding or subsequent embodiment, wherein the phase modulating element comprises a spatial light modulators (SLM).

The method or system of any preceding or subsequent embodiment, wherein the phase modulating element comprises a liquid deformable lens (LDL).

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the term “set” refers to a collection of one or more objects. Thus, for example, a set of objects can include a single object or multiple objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

TABLE 1
Key optics and parameters (enlarged in Figures)

| Optics | L1 | VL | L2 | L3 | L4 | L5 | L6 | L7 | L8 | CL |
|---|---|---|---|---|---|---|---|---|---|---|
| Key | f = | D = | f = | f = | f = | f = | f = | f = | f = | R = |
| parame- | 14 mm | 3.9 mm | 177.8 mn | 177.8 mn | 160 mn | 102.4 mn | 60 mm | 25 mm | 102.4 mn | 1.65 mm |
| ters | | | | | | | | | | |
| Comments | Output | Focus | Relay pupil to DM, | | Relay pupil to | | Relay pupil to | | beam | 3 mm in |
| | beam | Range | beam size on DM | | Scanner, | | mouse eye, | | size on | diameter, |
| | size: | : −5 to | is 3.5 mm | | beam size on | | beam size on | | is 2.24 | 0.25 mm |
| | 3.5 | +15 | | | scanner is 2.24 | | mouse pupil | | mm for | thickness |
| | mm | Diopters | | | mm | | is 0.93 mm | | US | 0 |
| | | | | | | | | | 1951 | Diopter |
| | | | | | | | | | resolution | power |
| | | | | | | | | | test | |
| | | | | | | | | | target | |
| | | | | | | | | | imaging | |

```
TABLE 2
Code for finding mirror configurations corresponding to brightest images.
clear;clc;close all
DataFolder=pwd;
A scan num=512;
fileinfo=dir(′Linear_Amp_FFT2X.tif′);
data=Read_Tiff_Stack(fileinfo.name);
for iii=1:size(data,3)
  temp=data(:,:,iii);
  imagesc(temp);
% max_temp=max(temp,[ ] ,1 );
  AVG_Max(iii)=mean (mean (temp (380:440,:),1));
end
load random_z.mat
[YYY, OOO] =sort(AVG_Max, ′descend′);
figure;plot (YYY);
Max_random_100=random_z(OOO (1:100),:);
opt=1;
if opt==1 %101010101010101010101010
teste=[Max_random_100, zeros (size (Max_random_100))]
;
Max_random_1000=reshape (teste′, 37, size (Max_random_
100, 1)*2)′;
elseif opt==2 %111111111111000000000000
  portion=Max_random_100(1:50, :);
Max_random_1000=[portion; zeros (size (portion))];
else
  Max_random_1000=random z(OOO (1:1000),:);
end
save Max_random_100 Max_random_100
cd(′F:\Code\My_IRIS_AO\2.5\VIs\Matlab ×64 IRISAO′)
;
% load optimized.mat
save Max_random_1000_7 Max_random_1000
cd(DataFolder);
```

TABLE 3
Code for testing optimal mirror configuration
histogram ring by ring.

```
clear;clc;close all
load Mirror_Configurations.mat
curr_path=pwd;
xlim_range=[−0.18,0.18];
hist_fit_option=′normal′;
%%%% for 1st ring segments
figure(′NumberTitle′, ′off′, ′Name′, This is the
10% higtogram for different ring segments');
subplot (2,2,1); hHist =histfit(
Sorted_random_z(1:100,1), 11, hist_fit_option
);xlim(xlim_range);title(′black *′,′Color′,′k′);%
histogram of 10%
% hold on;plot(hHist(1).XData,hHist(1).YData);
temp=Sorted_random_z(1:100,2:7);temp=temp(:);
subplot(2,2,2); hHist=histfit( temp,11,
hist_fit_option );xlim(xlim_range);title(′green
*′,′Color′,′g′);% histogram of 10%
temp=Sorted_random_z(1:100,8:19);temp=temp(:);
subplot(2,2,3); hHist =histfit(temp,11,
hist fit option );xlim(xlim range);title(′cyan
*′,′Color′,′c′);% histogram of 10%
temp=Sorted_random_z(1:100,20:end);temp=temp(:);
subplot(2,2,4); hHist =histfit(temp,11,
hist_fit_option );xlim(xlim_range);title(′blue
*′,′Color′,′b′);% histogram of 10% hold on; hHist =
histfit( Sorted_random_z(1:100,1),11, ′normal′ );
saveas (gcf, ′histo_ring_by_ring.tif′);
%%%% for individual segments
size rrr=100; % hegxon size
how_many_rrr=7;
pupil size=how_many_rrr*size_rrr; %in this case,
the pupil size pixel is fixed.
% Random_Amp=0.3;
[ my_center0 ] =cal_plot_hexgon_v2(
size_rrr,how_many_rrr/2 );
% hold on;
plot(my_center0(1,[2:7, 2]),my_center0(2, [2:7,2]),′
b--′,′linewidth′,2);
% hold on;
plot (my center0(1,[8:19,8]),my_center0(2, [8:19,8])
c--′,′linewidth′,2);
% hold on;
plot(my_center0(1,[20:end,20]),my_center0(2, [20:en
d,20]),′k--′,′linewidth′,2);
saveas(gcf, ′ring_location.tif′);
function [ my_center0 ] =cal_plot_hexgon_v2(
size rrr, how many rrr)
%CAL_PLOT_HEXGON_Summary of this function goes
here
% Detailed explanation goes here
theta_3_1=(0:60:300)*pi/180;
bbb1=sqrt(3\hat{ }2+1\hat{ }2−2*3*1*cos(pi/3));
bbb2=sqrt(3\hat{ }2+2\hat{ }2−2*3*2*cos(pi/3));
alpha1=acos((bbb1\hat{ }2+3\hat{ }2−1\hat{ }2)/(2*bbb1*3));
a1pha2=acos((bbb2A2+3\hat{ }2−2\hat{ }2)/(2*bbb2*3));
theta_3_2=(0:60:300)*pi/180+alpha1;
theta_3_3=(0:60:300)*pi/180+alpha2;
theta_3=[theta_3_1;theta_3_2;theta_3_3];
theta_3=theta_3(:)';
theta=
_3];
r_temp1=ones(1,6)*size_rrr*sqrt(3)/2;
r_temp2=[2*ones(1,6)*size_rrr*sqrt(3)/2;ones(1, 6)*
size rrr*3/2];
r_ccc=3*size_rrr*sqrt(3)/2;
r_aaa=size_rrr*sqrt(3)/2;
beta=60*pi/180;
r_temp3=[3*ones(1,6)*size_rrr*sqrt(3)/2;ones(1,6)*
(sqrt(r_cccA2]r_aaa\hat{ }2−
2*r_aaa*r_ccc*cos(beta)));ones(1,6)*(sqrt(r_ccc\hat{ }2+
4*r_aaaA2−2*2*r_aaa*r_ccc*cos(beta)))];
r=[0,r_temp1,r_temp2(:)',r_temp3(:)'];
xxx=r.*cos(theta)+size_rrr*how_many_rrr; %400
offset x
yyy=r.*sin(theta)+size_rrr*how_many_rrr; %300
offset y
figure;
plot(xxx(1),yyy(1),′k*′);hold on;
plot(xxx(2:7),yyy(2:7),′g*′);hold on;
plot(xxx(8:19),yyy(8:19),′c*1);hold on;
plot(xxx(20:end),yyy(20:end),′b*′);hold on;
colorbar;hold on;
% hold on; circle(
size_rrr*2.5,size_rrr*how_many_rrr,size_rrr*how_ma
ny_rrr,1000 );
% figure;
for iii=1:length(xxx)
hexagon(size_rrr/2,xxx(iii),yyy(iii),theta(iii));h
old on;
% hold
on;text(xxx(iii),yyy(iii),num2str(iii));
end
my center0=[xxx;yyy];
%
figure;plot(my_center0 (1,:),my_center0 (2,:), ′r*′);
% xlim([0,700]);ylim([0,700]);
end
function hexagon(cote,x0,y0,xita)
% cote=side size;,(x0,y0) exagon center
coordinates;
  beta=[30,90,150,210,270,330,390]*pi/180;
  x=cote*cos(beta);
  y=cote*sin(beta);
  RRR=[cos(xita),-
sin(xita);sin(xita),cos(xita)];
  tran=RRR*[x;y];
  new_x=tran(1, :);
  new_y=tran(2, :);
  x=x+x0;
  y=y+y0;
  plot(x,y,′r′,′Linewidth′,1);hold on;
% axis([x0−cote x0+cote y0−cote y0+cote]);
end
function [ ] =show mirror configuration(
Sorted_random_z,curr path )
% SHOW_MIRROR_CONFIGURATION
'Ë' ¦ ÏÔE¾ÓD1Ó'Ë°⁻ÊýμÄÕ^{a}Ò^{a}
% ′Ë' ¦ ÏÔÊ3/4ÏeÏ,EμÃ÷
size_rrr=100; % hegxon size
how_many_rrr=7;
pupil_size=how_many_rrr*size_rrr; %in this case,
the pupil size pixel is fixed.
% Random Amp=0.3;
[ my_center0 ] =cal_plot_hexgon(
size_rrr,how_many_rrr/2 );
saveas(gcf,′segment_num.tif′);
for iii=1:size(Sorted_random_z, 1)
  [ Wavefront ] =cal_plot_wavefront(
pupil_size, Sorted_random_z(iii,:),size_rrr,my_cent
er0 );
  figure;mesh(Wavefront);colorbar;caxis([−
0.15,0.15]);
  % set(gca,′xtick′,[ ]);
  % set(gca,′ytick′,[ ]);
  % axis equal
  az=0;
  el=90;
  view(az, el);
saveas(gcf,strcat(curr_path,′/Top100_MirrorConfig/
′,num2str(iii),′.tif′));
  close all;
end
end
```

What is claimed is:

1. A method of speckle free optical coherence tomographic imaging, the method comprising:
(a) providing a confocal coherent detection system with an entrance aperture;
(b) controlling modulation of entrance aperture aberrations;
(c) generating a set of optimally aberrated point-spread functions (PSFs) in a sample; and
(d) producing an image of the sample using the generated optimally aberrated point-spread functions.

2. The method of claim 1, wherein said modulation of entrance aperture aberrations is controlled with adaptive optics elements selected from the group of elements consisting of a deformable mirror, a segmented mirror and a spatial light modulator.

3. The method of claim 1, wherein generation of said set of optimally aberrated point-spread functions (PSFs) in a sample comprises:
(a) modulating a phase inside the imaging system pupil aperture with a segmented deformable mirror to produce minor perturbations in the point spread function (PSF) and create un-correlated speckle patterns between B-scans;
(b) applying an averaging technique to the patterns to wash out speckle but maintain structures; and
(c) searching for optimally aberrated point-spread functions.

4. The method of claim 1, further comprising:
(a) acquiring an interlaced B-scan, an adaptive optics-optical coherence tomography (AO-OCT) scan, and an aperture phase modulation-adaptive optics-optical coherence tomography (APM-AO-OCT) B-scan; and
(b) producing standard and speckle free images from said scans.

5. The method of claim 1, further comprising:
(a) acquiring speckle free aperture phase modulation-adaptive optics-optical coherence tomography (APM-AO-OCT) images of a biological sample;
(b) acquiring intrinsic sample motion images;
(c) comparing intrinsic sample motion images and speckle free APM-AO-OCT images; and
(d) identifying static and dynamic structures of the biological sample.

6. The method of claim 1, further comprising:
(a) acquiring serial aperture phase modulation-adaptive optics-optical coherence tomography (APM-AO-OCT) interlaced B-scans of a sample; and
(b) building an optical coherence tomography (OCT) volume of the sample from the APM-AO-OCT interlaced B-scans of the sample with slow data acquisition or static sample scans

7. The method of claim 1, further comprising:
(a) acquiring serial volumetric aperture phase modulation-adaptive optics-optical coherence tomography (APM-AO-OCT) interlaced B-scans of a sample; and
(b) building APM-AO-OCT interlaced volume of the sample from the volumetric APM-AO-OCT interlaced B-scans of the sample with fast data acquisition or moving sample scans.

8. A method of speckle free optical coherence tomographic imaging, the method comprising:
(a) providing an optical coherence tomographic system with segmented wavefront correctors;
(b) deforming the segmented wavefront correctors to maintain lateral resolution while varying point-spread functions (PSFs); and
(c) producing an image of a sample using generated optimum point-spread functions.

9. The method of claim 8, further comprising:
(a) randomly deforming the segmented wavefront correctors to produce uncorrelated speckle patterns;
(b) searching for optimum point-spread functions; and
(c) averaging optimized sets of aperture phase modulation-adaptive optics-optical coherence tomography (APM-AO-OCT) B-scans.

10. The method of claim 8, further comprising:
(a) optimizing a mirror segment displacement range; and
(b) selecting a subset of mirror configurations within the optimum range to satisfy triple constraints of greatly reducing speckle noise while simultaneously maximally preserving resolution and signal strength.

11. A method for generating a set of random mirror configurations for use in optimization of an aperture phase modulation-adaptive optics-optical coherence tomography (APM-AO-OCT) signal where a deformable mirror (DM) having mirror segments is used, the method comprising:
(a) performing an optical coherence tomography (OCT) scan and acquiring an image;
(b) adding random phase displacement for each mirror segment;
(c) recording image and mirror configurations corresponding to the phase displacement; and
(d) repeating steps (b) and (c) until a set of presets are exhausted.
